(19) United States
(12) Patent Application Publication
(10) Pub. No.: US 2014/0051602 A1
(43) Pub. Date: Feb. 20, 2014

(54) DETECTING ANALYTES USING LIPID MULTILAYER GRATINGS WITH ION CHANNELS

(71) Applicant: FLORIDA STATE UNIVERSITY RESEARCH FOUNDATION, Tallahassee, FL (US)

(72) Inventor: STEVEN LENHERT, TALLAHASSEE, FL (US)

(73) Assignee: FLORIDA STATE UNIVERSITY RESEARCH FOUNDATION, Tallahassee, FL (US)

(21) Appl. No.: 14/060,807

(22) Filed: Oct. 23, 2013

Related U.S. Application Data
(63) Continuation-in-part of application No. PCT/IB2013/055884, filed on Jul. 17, 2013.
(60) Provisional application No. 61/672,505, filed on Jul. 17, 2012.

(51) Int. Cl. G01N 33/50

(52) U.S. Cl. CPC G01N 33/5076
USPC 506/12; 506/15

(57) ABSTRACT

A method comprising the following step: determining that one or more analytes are present in a fluid to which an array of lipid multilayer gratings has been exposed based on scattered light detected by a detector, wherein each lipid multilayer grating of the array of lipid multilayer gratings comprises iridescent lipid multilayer nanostructures, wherein the scattered light is formed by scattering one or more incident lights by the array of lipid multilayer gratings, and wherein the one or more lipid multilayer gratings comprise ion channels that are activated by the one or more analytes.

### CROSS-REFERENCE TO RELATED APPLICATIONS

This continuation-in-part application claims benefit of priority to International Patent Application No. PCT/IB2013/055884 to Lenhert, entitled “SURFACE SUPPORTED LIPOSOME NANORRAYS AS BIOMIMETIC SENSORS,” filed Jul. 17, 2013, which in turn claims the benefit of priority to U.S. Provisional Patent Application No. 61/672,505, entitled “SURFACE SUPPORTED LIPOSOME NANOARRAYS AS BIOMIMETIC SENSORS,” filed Jul. 17, 2012. The entire contents and disclosure of these two applications are incorporated herein by reference.

This application makes reference to the above-cited patent application and the following U.S. Patent Applications: U.S. Provisional Patent Application No. 61/383,775, entitled “HIGH THROUGHPUT OPTICAL QUALITY CONTROL OF PHOSPHOLIPID MULTILAYER FABRICATION VIA DIP PEN NANOLITHOGRAPHY (DPN),” filed Sep. 17, 2010. U.S. Provisional Patent Application No. 61/387,764, entitled “NOVEL DEVICE FOR DETECTING AND ANALYZING AQUEOUS SAMPLES,” filed Sep. 21, 2010. U.S. Provisional Patent Application No. 61/387,550, entitled “LIPID MULTILAYER GRATINGS,” filed Sep. 29, 2010. U.S. Provisional Patent Application No. 61/387,556, entitled “LIPID MULTILAYER GRATINGS FOR SEMISYNTHETIC QUORUM SENSORS,” filed Sep. 29, 2010. U.S. Provisional Patent Application No. 61/451,619, entitled “IRIDESCENT SURFACES AND APPARATUS FOR REAL TIME MEASUREMENT OF LIQUID AND CELLULAR ADHESION,” filed Mar. 11, 2011. U.S. Provisional Patent Application No. 61/451,635, entitled “METHODS AND APPARATUS FOR LIPID MULTILAYER PATTERNING,” filed Mar. 11, 2011. U.S. Provisional Patent Application No. 61/501,298, entitled “LIPOSOME MICROARRAY SURFACE AND THEIR USE FOR CELL CULTURE SCREENING,” filed Jun. 27, 2011. U.S. patent application Ser. No. 13/234,540, entitled “OPTICAL METHOD FOR MEASURING HEIGHT OF FLUORESCENT PHOSPHOLIPID FEATURES FABRICATED VIA DIP-PEN NANOLITHOGRAPHY,” filed Sep. 11, 2011. U.S. patent application Ser. No. 13/238,498, entitled “INTEGRATED DEVICE FOR ANALYZING AQUEOUS SAMPLES USING LIPID MULTILAYER,” filed Sep. 21, 2011. U.S. patent application Ser. No. 13/248,250, entitled “SEMI-SYNTHETIC QUORUM SENSORS, filed Sep. 29, 2011. U.S. Provisional Patent Application No. 60/570,490, entitled “LIPID MULTILAYER MICROARRAYS FOR IN VITRO LIPOSOMAL DRUG DELIVERY AND SCREENING,” filed Dec. 14, 2011. U.S. Provisional Patent Application No. 61/577,834, entitled “HIGH THROUGHPUT SCREENING METHOD AND APPARATUS,” filed Dec. 20, 2011. U.S. Provisional Patent Application No. 61/577,910, entitled “NANOSTRUCTURED LIPID MULTILAYER FABRICATION AND DEVICES THEREOF,” filed Dec. 20, 2011. U.S. Provisional Patent Application No. 61/671,214, entitled “SCALABLE LIPOSOME MICROARRAY SCREENING, filed Jul. 13, 2012. The entire contents and disclosures of these patent applications are incorporated herein by reference.

### BACKGROUND

1. Field of the Invention

The present invention relates to the use of photonic sensors.

2. Related Art

Current methods for identifying bacteria grown in culture, and detecting impurities in food and water have various deficiencies.

### SUMMARY

According to a first broad aspect, the present invention provides a method comprising the following step: (a) determining that one or more analytes are present in a liquid to which an array of lipid multilayer gratings has been exposed based on scattered light detected by a detector, wherein each lipid multilayer grating of the array of lipid multilayer gratings comprises iridescent lipid multilayer nanostructures, wherein the scattered light is formed by scattering one or more incident lights by the array of lipid multilayer gratings, wherein the scattered light is formed while the lipid multilayer gratings are immersed in the liquid, and wherein the one or more lipid multilayer gratings comprise ion channels that are activated by the one or more analytes.

According to a second broad aspect, the present invention provides a product comprising: an array of lipid multilayer gratings on a substrate, wherein each lipid multilayer grating of the array of lipid multilayer gratings comprises iridescent lipid multilayer microstructures, and wherein each lipid multilayer structure of the lipid multilayer microstructures each comprise one or more ion channels on a surface of the lipid multilayer structure.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a schematic illustration of lipids being transferred from the tip of an atomic force microscope (AFM) to a solid surface to form surface-supported multilayers.

FIG. 2 is a reflection-mode optical micrograph of multilayer squares patterned on plasma-oxidized silicon at various scan speeds.

FIG. 3 is a graph showing the height of phospholipid multilayers and the corresponding number of bilayer stacks measured by AFM, plotted as a function of scan speed on a logarithmic scale at two different relative humidities.

FIG. 4 is a diagram showing the chemical and supramolecular structures of liposomes and surface-supported lipid nanostructures.

FIG. 5 is a merged image where the top portion is a schematic drawing of different tips in a parallel array integrating different inks on a surface. The bottom portion is a fluorescence micrograph of multicomponent phospholipid patterns with neighboring-dot spacing of 2 μm.

FIG. 6 is a large-area fluorescence micrograph of a row of “FSU” patterns of different heights, seen as different fluorescence intensities.

FIG. 7 is an enlargement of the area highlighted in FIG. 6.

FIG. 8 is a fluorescence micrograph where the structure heights are determined by means of calibrated fluorescence intensities.

FIG. 9 is an AFM image of the area shown in FIG. 7.

FIG. 10 is an AFM image showing the height measurement confirming the measurement in FIG. 9.

FIG. 11 is a schematic illustration of the technique used to fabricate lipid multilayer gratings.

FIG. 12 is a graph showing the application of functional lipid multilayer gratings as biosensors, where optical diffraction is monitored as proteins bind to the oil-water interface, and the response is correlated with protein concentration.

FIG. 13 is a diagram showing the theoretical relation between the height of adherent lipid droplet height with the three interfacial energies involved.

FIG. 14 is a schematic illustration of lipid multilayer stamping, wherein different lipid inks are microarrayed onto the surface.

FIG. 15 is an image of the red diffraction obtained from stamped DPPC.

FIG. 16 is an optical micrograph with surface enhanced ellipsometric contrast (SEEC) imaging of the area indicated with a white square in FIG. 16, showing DPPC grating lines over a large area.

FIG. 17 is an AFM height image of the region indicated with a white square in FIG. 17.

FIG. 18 is an image of the line trace of gratings in FIG. 17 that show an average height of 110±10 nm.

FIG. 19 is a table listing the lipids that are integrated in the multicomponent lipid arrays in this present invention.

FIG. 20 is a schematic illustration of lipid spreading which is a mechanism by which the lipid multilayers change shape and therefore their shape-dependent optical properties on which the biosensor is based.

FIG. 21 is a graph showing the change in area spreading of lipids as a function different time, measured at different pH values.

FIG. 22 is a plot of the spreading rate (slopes of the lines in FIG. 21) as a function of pH.

FIG. 23 is a schematic illustration of the amplification of a chemical signal for detection of a catalyst in an aqueous solution using a reaction within a lipid multilayer grating according to one embodiment of the present invention.

FIG. 24 is a schematic illustration of the amplification of a chemical signal for detection of an analyte in an aqueous solution using a reaction within a lipid multilayer grating according to one embodiment of the present invention.

FIG. 25 is a schematic illustration of an analyte detection process according to one embodiment of the present invention.

FIG. 26 is a schematic illustration of enzyme-linked immunosorbent assay (ELISA) process according to one embodiment of the present invention.

FIG. 27 is a schematic illustration of assay process according to one embodiment of the present invention.

### DETAILED DESCRIPTION OF THE PREFERRED EMBODIMENTS

### Definitions

Where the definition of terms departs from the commonly used meaning of the term, applicant intends to utilize the definitions provided below, unless specifically indicated.

For purposes of the present invention, it should be noted that the singular forms, “a,” “an” and “the” include reference to the plural unless the context as herein presented clearly indicates otherwise.

For purposes of the present invention, directional terms such as “top,” “bottom,” “upper,” “lower,” “above,” “below,” “left,” “right,” “horizontal,” “vertical,” “up,” “down,” etc., are used merely for convenience in describing the various embodiments of the present invention. The embodiments of the present invention may be oriented in various ways. For example, the diagrams, apparatuses, etc., shown in the drawing figures may be flipped over, rotated by 90° in any direction, reversed, etc.

For purposes of the present invention, a value or property is “based” on a particular value, property, the satisfaction of a condition, or other factor, if that value is derived by performing a mathematical calculation or logical decision using that value, property or other factor.

For purposes of the present invention, the term “analyte” refers to the conventional meaning of the term “analyte,” i.e., a substance or chemical constituent of a sample that is being detected or measured in a sample. In one embodiment of the present invention, a sample to be analyzed may be an aqueous sample, but other types of samples may also be analyzed using a device of the present invention. A contaminant is one type of analyte.

For purposes of the present invention, the term “aqueous analyte” refers to a substance dissolved in or suspended in water.

For purposes of the present invention, the term “array” refers to a one-dimensional or two-dimensional set of microstructures. An array may be any shape. For example, an array may be a series of microstructures arranged in a line, such as an array of squares. An array may be arranged in a square or rectangular grid. There may be sections of the array that are separated from other sections of the array by spaces. An array may have other shapes. For example, an array may be a series of microstructures arranged in a series of concentric circles, in a series of concentric squares, a series of concentric triangles, a series of curves, etc. The spacing between sections of an array or between microstructures in any array may be regular or may be different between particular sections or between particular pairs of microstructures. The microstructure arrays of the present invention may be composed of microstructures having zero-dimensional, one-dimensional or two-dimensional shapes. The microstructures having two-dimensional shapes may have shapes such as squares, rectangles, circles, parallelograms, pentagons, hexagons, irregular shapes, etc.

For purposes of the present invention, the term “away” refers to increasing the distance between two aligned objects. For example, a contact controlling positioning device may be used to move: a stamp away from an ink palette, an ink palette away from a stamp, a stamp away from a substrate, a substrate away from a stamp, etc.

For purposes of the present invention, the term “biomolecule” refers to the conventional meaning of the term biomolecule, i.e., a molecule produced by or found in living cells, e.g., a protein, a carbohydrate, a lipid, a phospholipid, a nucleic acid, etc.

For purposes of the present invention, the term “calcium channel” refers to an ion channel that displays permeability to calcium ions.

For purposes of the present invention, the term “camera” refers to any type of camera or other device that senses light intensity. Examples of cameras include digital cameras, scanners, charged-coupled devices, CMOS sensors, photomultiplier tubes, analog cameras such as film cameras, etc. A camera may include additional lenses and filters such as the lenses of a microscope apparatus that may be adjusted when the camera is calibrated.

For purposes of the present invention, the term “contacting surface” refers to a surface of a stamp that contacts a surface onto which a pattern comprising lipid ink is to be printed.

For purposes of the present invention, the term “contaminant” refers to any biological, chemical, physical or radiological substance in a fluid that in sufficient concentration may adversely affect living organism.

For purposes of the present invention, the term “control fluid” refers to a fluid that is pure or that contains known concentrations of one or more analytes. A control fluid may be used in determining a standard reading for a particular type of array of iridescent lipid multilayer nanostructures by a particular type of detector. A control fluid may also be used to determining a base reading for light that is scattered by an iridescent array and detected by a detector prior to exposing the array to a fluid containing one or more analytes. The presence and/or concentration of the one or more analytes in the fluid may be determined by comparing the light scattered by the array and detected by the detector after the exposure of the array to the fluid containing the one or more analytes to the base reading for the control fluid.

For purposes of the present invention, the term “controlled environment chamber” refers to a chamber in which temperature and/or pressure and/or humidity can be controlled.

For purposes of the present invention, the term “dehydrated lipid multilayer grating” refers to a lipid multilayer grating that is sufficiently low in water content that it is no longer in fluid phase.

For purposes of the present invention, the term “detector” refers to any type of device that detects or measures light. A camera is a type of detector.

For purposes of the present invention, the term “dot” refers to a microstructure that has a zero-dimensional shape.

For purposes of the present invention, the term “drug” refers to a material that may have a biological effect on a cell, including but not limited to small organic molecules, inorganic compounds, polymers such as nucleic acids, peptides, saccharides, or other biologic materials, nanoparticles, etc.

For purposes of the present invention, the term “fluid analyte” refers to any type of analyte in a fluid. An aqueous analyte is one type of fluid analyte.

For purposes of the present invention, the term “fluorescence” refers to the conventional meaning of the term fluorescence, i.e., the emission of light by a substance that has absorbed light or other electromagnetic radiation of a different wavelength.

For purposes of the present invention, the term “fluorescent” refers to any material or mixture of materials that exhibits fluorescence.

For purposes of the present invention, the term “fluorescent dye” refers to any substance or additive that is fluorescent or imparts fluorescence to another material. A fluorescent dye may be organic, inorganic, etc.

For purposes of the present invention, the term “fluorescent microstructure” refers to a microstructure that is fluorescent. A fluorescent microstructure may be made of a naturally fluorescent material or may be made of a nonfluorescent material, such as a phospholipid, doped with a fluorescent dye.

For purposes of the present invention, the term “fluorescent nanostructure” refers to a nanostructure that is fluorescent. A fluorescent nanostructure may be made of a naturally fluorescent material or may be made of a nonfluorescent material, such as a phospholipid, doped with a fluorescent dye.

For purposes of the present invention, the term “fluid” refers to a liquid or a gas.

For purposes of the present invention, the term “freezing by dehydration” refers to removal of residual water content, for instance by incubation in an atmosphere with low water content, for instance a vacuum (<50 mbar) or at relative humidity below 40% (at standard temperature and pressure).

For purposes of the present invention, the term “grating” refers to an array of dots, lines, or a 2D shape that are regularly spaced at a distance that causes coherent scattering of incident light.

For purposes of the present invention, the term “groove” refers to an elongated recess in a stamp. A groove is not limited to a linear groove, unless clearly specified otherwise in the description below. The dimensions of a groove may change depending on the depth of the groove. For example, a groove may be wider at the top of the groove than at the bottom of the groove, such as in a V-shaped groove.

For purposes of the present invention, the term “groove pattern” refers to the pattern made by one or more grooves of a stamp.

For purposes of the present invention, the term “height” refers to the maximum thickness of the microstructure on a substrate, i.e., the maximum distance the microstructure projects above the substrate on which it is located.

For purposes of the present invention, the term “high humidity atmosphere” refers to an atmosphere having a relative humidity of 40% or greater.

For purposes of the present invention, the term “ion channels” refer to a protein, polymer or other molecular entity that gates the flow of ions into and/or out of lipid multilayers or across a lipid bilayer. In biological membranes, ion channels gate the flow of ions across the biological membrane.

For purposes of the present invention, the term “iridescent” refers to any structure that scatters light.

For purposes of the present invention, the term “iridescent microstructure” refers to a microstructure that is iridescent.

For purposes of the present invention, the term “iridescent nanostructure” refers to a nanostructure that is iridescent.

For purposes of the present invention, the term “irregular pattern” refers to a pattern of ridges and recesses that are not organized in a specific geometric pattern. For example, ridges and or recesses printed to resemble a picture of a human face, a picture of a leaf, a picture of an ocean wave, etc. are examples of irregular patterns. Using photolithography, almost any type of pattern for recesses and/or ridges may be formed in a stamp of the present invention.

For purposes of the present invention, the term “light,” unless specified otherwise, refers to any type of electromagnetic radiation. Although, in the embodiments described below, the light that is incident on the gratings or sensors is visible light, the light that is incident on the gratings or sensors of the present invention may be any type of electromagnetic radiation, including infrared light, ultraviolet light, etc., that may be scattered by a grating or sensor. Although, in the embodiments described below, the light that is scattered from the gratings or sensors and detected by a detector is visible light, the light that is scattered by a grating or sensor of the present invention and detected by a detector of the present invention may be any type of electromagnetic radiation, including infrared light, ultraviolet light, etc. that may be scattered by a grating or sensor.

For purposes of the present invention, the term “light source” refers to a source of incident light that is scattered by a grating or sensor of the present invention. In one embodiment of the present invention, a light source may be part of a device of the present invention. In one embodiment a light source may be light present in the environment of a sensor or grating of the present invention. For example, in one embodiment of the present invention a light source may be part of a device that is separate from the device that includes the sensors and detector of the present invention. A light source may even be the ambient light of a room in which a grating or sensor of the present invention is located. Examples of a light source include a laser, a light-emitting diode (LED), an incandescent light bulb, a compact fluorescent light bulb, a fluorescent light bulb, etc.

For purposes of the present invention, the term “line” refers to a “line” as this term is commonly used in the field of nanolithography to refer to a one-dimensional shape.

For purposes of the present invention, the term “lipid” refers to hydrophobic or amphipilic molecules, including but not limited to biologically derived lipids such as phospholipids, triacylglycerols, fatty acids, cholesterol, or synthetic lipids such as surfactants, organic solvents, oils, etc.

For purposes of the present invention, the term “lipid ink” refers to any material comprising a lipid applied to a stamp.

For purposes of the present invention, the term “lipid multilayer” refers to a lipid coating that is thicker than one molecule.

For purposes of the present invention, the term “lipid multilayer grating” refers to a grating comprising lipid multilayers.

For purposes of the present invention, the term “lipid multilayer structure” refers to a structure comprising one or more lipid multilayers. A lipid multilayer structure may include a dye such as a fluorescent dye.

For purposes of the present invention, the term “low humidity atmosphere” refers to an atmosphere having a relative humidity of less than 40%.

For purposes of the present invention, the term “lyotropic” refers to the conventional meaning of the term “lyotropic,” i.e., a material that forms liquid crystal phases because of the addition of a solvent.

For purposes of the present invention, the term “microarray” refers to an array of microstructures.

For purposes of the present invention, the term “microfabrication” refers to the design and/or manufacture of microstructures.

For purposes of the present invention, the term “microstructure” refers to a structure having at least one dimension smaller than 1 mm. A nanostructure is one type of microstructure.

For purposes of the present invention, the term “nanoarray” refers to an array of nanostructures.

For purposes of the present invention, the term “nanofabrication” refers to the design and/or manufacture of nanostructures.

For purposes of the present invention, the term “neat lipid ink” refers to a lipid ink consisting of a single pure lipid ink.

For purposes of the present invention, the term “nanostructure” refers to a structure having at least one dimension on the nanoscale, i.e., a dimension between 0.1 and 100 nm.

For purposes of the present invention, the term “patterned substrate” refers to a substrate having a patterned array of lipid multilayer structures on at least one surface of the substrate.

For purposes of the present invention, the term “palette” refers to a substrate having one or more lipid inks that are made available to be picked up or drawn into the recesses or other topographical or chemical features of a stamp. The one or more lipid inks may be located in recesses, inkwells, etc. in the palette, or deposited onto a flat palette.

For purposes of the present invention, the term “palette spot” refers to a single spot of lipid link on a palette. A palette spot may be any shape.

For purposes of the present invention, the term “plurality” refers to two or more. So an array of microstructures having a “plurality of heights” is an array of microstructures having two or more heights. However, some of the microstructures in an array having a plurality of heights may have the same height.

For purposes of the present invention, the term “reagent” refers to a chemical or biological material that reacts with an analyte.

For purposes of the present invention, the term “recess” refers to a recess of any size or shape in a stamp. A recess may have any cross-sectional shape such as a line, a rectangle, a square, a circle, an oval, etc. The dimensions of a recess may change depending on the depth of the recess. For example, a recess may be wider at the top of the recess than at the bottom of the recess, such as in a V-shaped recess. An example of a recess is a groove.

For purposes of the present invention, the term “recess pattern” refers to the pattern made by one or more recesses of a stamp.

For purposes of the present invention, the term “regular pattern” refers to a pattern of ridges and recesses organized in a specific geometric pattern. For example, a series of parallel recesses and/or lines is one example of a regular pattern. One or more arrays of ridges and recesses arranged in a square, a circle, an oval, a star, etc. is another example of a regular pattern.

For purposes of the present invention, the term “patterned array” refers to an array arranged in a pattern. A patterned array may comprise a single patterned array of lipid multilayer structures or two or more patterned arrays of lipid multilayer structures. Examples of patterned arrays of lipid multilayer structures are a patterned array of dots, a patterned array of lines, a patterned array of squares, etc.

For purposes of the present invention, the term “printing” refers to depositing a material, such as lipid ink, on a substrate.

For purposes of the present invention, the term “removing” refers to removing two objects from each other by moving one or both objects away from each other. For example, a stamp may be removed from a palette or substrate by moving the stamp away from the palette or substrate, by moving the palette or substrate away from the stamp or by moving both the stamp and the palette or substrate away from each other.

For purposes of the present invention, the term “ridge” refers to any raised structure. A ridge is not limited to a linear ridge, unless clearly specified otherwise in the description below. A ridge may have any cross-sectional shape such as a line, a rectangle, a square, a circle, an oval, etc. The dimensions of a ridge may change depending on the depth of a neighboring groove. For example, a ridge may be wider at the bottom of the ridge than at the top of the ridge, such as in a V-shaped ridge. A ridge may constitute the entire contacting surface of a stamp after recesses have been formed, etched, etc. into the stamp.

For purposes of the present invention, the term “scattering” and the term “light scattering” refer to the scattering of light by deflection of one or more light rays from a straight path due to the interaction of light with a grating or sensor. One type of interaction of light with a grating or sensor that results in scattering is diffraction.

For purposes of the present invention, the term “sensor” and the term “sensor element” are used interchangeably, unless specified otherwise, and refer to a material that may be used to sense the presence of one or more analytes.

For purposes of the present invention, the term “square” refers to a microstructure that is square in shape, i.e., has a two-dimensional shape wherein all sides are equal.

For purposes of the present invention, the term “stamped spot” refers to an area of a patterned surface of lipid nanostructures that originates from a single palette spot on an ink palette used as a source of lipid ink by stamp in depositing the lipid nanostructure. A stamped spot may be any shape.

For purposes of the present invention, the term “standard reading” refers to the readings obtained by a particular type of detector for light scattered by an array or by arrays iridescent lipid multilayer nanostructures similar to or identical to an array that has been exposed to a particular fluid.

### Description

Lipids molecules that form the structural and functional basis of biological membranes, which are highly multifunctional interfaces that have evolved in nature and are essential to all known forms of life. For example, the outer surface of a single cell (or cell membrane) is composed of a lipid bilayer that can be viewed as a two-dimensional organic phase where lipophilic molecules carry out their various functions. The cell membrane is capable of specifically detecting thousands of different molecules and initiating specific signaling cascades within the cell—i.e., signal transduction. Biological lipids have therefore been employed biosensor functionalization.⁶ Surface-supported lipid bilayer membranes can be formed on hydrophilic surfaces by several methods.⁷⁻⁸ The most commonly used method of forming lipid bilayer membranes on hydrophilic surfaces is known as vesicle fusion, in which vesicles formed in solution are incubated with a hydrophilic surface where they fuse and form a contiguous bilayer.⁹ Biological membranes have been shown to be inhomogeneous fluids with local domains where different proteins cluster, especially in the cases where a cell touches a nearby surface (e.g., another cell or the extracellular matrix).^{10,11,12} One approach to mimicking these types of cell-membrane microdomains is to pattern a substrate by standard photolithography or electron-beam lithography and then to form a lipid bilayer on that surface by means of vesicle fusion.¹³ The lithographic pattern forms diffusion barriers that corral the lipids into artificial domains within which they can freely diffuse. Culturing living cells on these patterned lipid bilayers has provided insight into cell-cell communication.^{14,15} Hydration-induced spreading of dehydrated lipid multilayers is another method for the formation of surface-supported lipid bilayers;^{16,17,18,19} it allows various lipids of different identities to be integrated onto the same surface.^{20,21} Other approaches to patterning of supported lipid bilayers include direct photolithography,^{12,22} microcontact printing,^{23,24} micropipetting,²⁵ and pin spotting.²⁶ Once supported on surfaces, lipid bilayers can be readily characterized by means of advanced fluorescence microscopy^{7,8} and atomic force microscopy (AFM).²⁷

Liposomes (or vesicles) are lipid particles that are three-dimensional, self-organized, nanostructured and widely used as drug- and gene-delivery vehicles. They can be created in a variety of different sizes (small, large, and giant) and forms (lamellar and multilamellar) and are typically characterized by dynamic light scattering and transmission electron microscopy.^{28,29,30} Antibodies and antibody fragments have been coupled to liposomes by means of maleimide coupling to allow specific delivery to certain cell types.^{31,32} Liposomes are also used in research as nanoscale reaction vessels,⁵ biosensors,³³ and artificial cells.^{34,35} For example, liposome adhesion to surfaces has been used as a model system for cell adhesion.³⁴ Self-replication of nucleic acids^{36,37} and protein expression³⁸ have been carried out within liposomes and have provided insight into the conditions under which life may have originated as well as progress toward a completely synthetic cell.^{39,40}

Microarrays have been very successful in biotechnology for screening purposes. In the case of DNA, for example, the microarray has allowed massively parallel experiments to be carried out on a single chip.⁴¹ Because tens to hundreds of thousands of pieces of data can be generated from a single sample at a reasonable cost, gene-expression analysis has become standard practice in biology laboratories. Similarly, protein microarrays are being developed for the screening of protein function,⁴² and microarrays of different types of lipids have been proposed for molecular screening applications. Spotting techniques are typically used to create arrays of lipid bilayers that are composed of different lipid materials on a surface that allows lipid-bilayer formation.^{43,44}

Surface patterning methods have a long history and are currently well developed for the microelectronics industry. Dip-pen nanolithography (DPN) is a method developed by Chad Mirkin (a mentor of the PI) that is now widely used for the rapid prototyping of novel nanostructured surfaces.^{45,46,47,48} DPN uses the tip from an AFM to deliver materials to a surface in a direct writing process, and it can fabricate arbitrary structures from a variety of molecular inks. The use of masks is not required, and sub-100-nm resolution can be achieved.⁴⁸ DPN is also capable of high throughput when carried out with parallel tip arrays.^{49,50} Similar approaches to nano- and microsurface patterning include soft lithography⁵¹ and polymer pen lithography.⁵²

Optical biosensors typically function by coupling the binding of an analyte of interest to a biofunctionalized transducer converts the binding event to an optical readout mechanism. Surface Plasmon Resonance (SPR) is a successful example where changes in the interaction with light and a biofunctionalized metal surface or particle upon analyte binding can be used as a sensing mechanism. Grating-based biosensors are another approach where a spectral change is detected upon analyte binding to the surface of a biofunctionalised diffraction grating.^{53,54,55}

A strategy for detecting compounds and qualities of complex mixtures is to use pattern recognition to detect patterns of binding events to sensor arrays, each of which may not be specific for a particular analyte or quality, but collectively can provide more information than individual sensors.⁵⁶ An analogy can be drawn between this approach and that taken by the mammalian olfactory system which contains about 1000 different receptor genes, each of which is not highly specific, but collectively produce a binding pattern that can be recognized by the nervous system to allow highly precise identification of useful qualities of complex mixtures. When this approach is used in combination with photonic sensors it is referred to as a “photonic nose.” This approach has been used to successfully identify bacteria grown in culture, and for quality control of food and water.

In one embodiment, the present invention employs microstructures and/or nanostructures formed on surfaces from biological lipids as biomimetic sensors. Lipids have evolved in nature to enable massively parallel sensing of biological agents by means of signal transduction, where a signaling molecule (i.e., analyte) typically binds to a receptor in a lipid membrane that in turn induces chemical reactions within the cell that lead to signal amplification and eventually a cellular response. Liposome nanoarrays may be used to integrate lipid biochemical functions with the dynamic compartmentalization properties of lipids.^{1,2,3} In particular, when lipid structures are formed on surfaces with thicknesses between 10-100 nanometers, and lateral dimensions on the scale of the wavelengths of visible light, the structures become iridescent which allow chemical interactions with these objects to be detected optically in a label free manner.¹

In one embodiment of the present invention, the innate biocompatibility of liposome microarrays are combined with their nanostructure dependent optical properties in order to enable novel biomimetic sensor array technology.

In one embodiment, the present invention provides a method of making iridescent arrays comprising multiple lipids.

In one embodiment, the present invention provides a method for determining the relationship between lipid composition, optical properties, and environmental conditions such as pH, salt concentration, and temperature.

In one embodiment, the present invention provides a method of signal amplification by means of confined chemical reactions.

In one embodiment, arrays of the present invention may be formed using lipid multilayer stamping, which is a scalable method of lipid nanomanufacturing.⁷⁰

In one embodiment, the present invention provides a method of environmental monitoring, such as determining water quality, with an array of lipid multilayer nanostructures using the concept of a photonic nose. For example, arrays of the present invention may be used to detect heavy metals and Pharmaceuticals and Personal Care Products (PPCPs) in wastewater.

In one embodiment, the present invention provides iridescent an array of lipid multilayer nanostructures that function as optical sensors capable of mimicking natural sensing mechanisms of cells in the mammalian olfactory system, i.e., as functioning as a photonic nose.

The present invention may be used in applications such as industrial processes in nanofabrication, sensor development, medicine, agriculture, environmental monitoring, and general sustainability. For example, massively parallel sensor arrays made possible by the proposed work would have immediate applications for

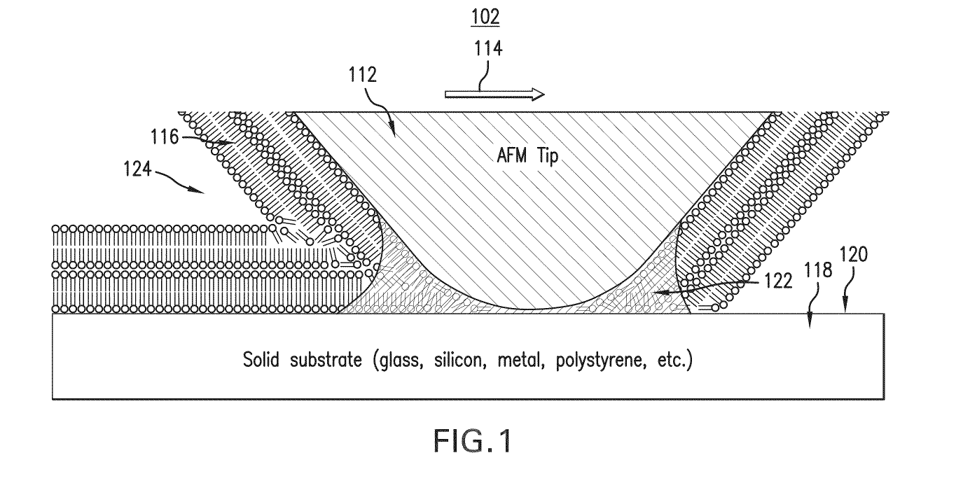

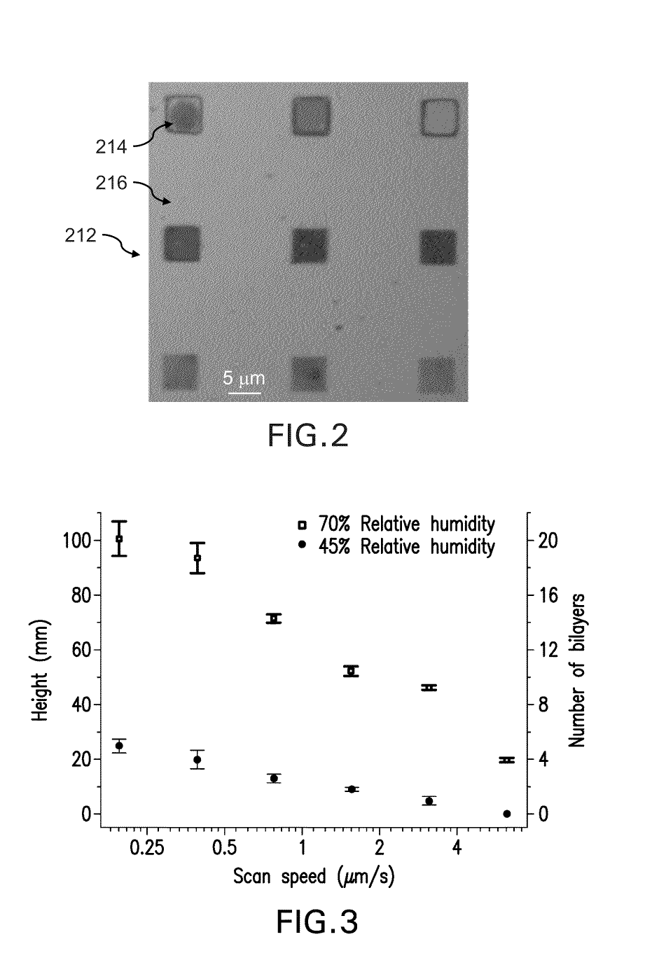

FIG. 1 is a schematic drawing of a lipid DPN process 102. An AFM tip 112 writing in a direction shown by arrow 114 coated with a phospholipid ink 116 is placed in contact with a substrate 118 so that the ink transfers from tip 112 to surface 120, forming surface-supported lipid multilayer patterns 122. In contrast to other molecular inks commonly used in DPN, phospholipid inks are deposited as multilayers; when these layers are thicker than roughly 20 nm and may be readily observed with an optical microscope, as shown in FIG. 2. It was discovered that during the DPN process, air humidity in a surrounding environment 124 in which DPN process 102 is carried out may be used to control the fluidity of DOPC. At high relative humidity (75%), DOPC becomes fluid and readily flows between the AFM tip and the substrate, whereas at lower relative humidity (45%), it becomes more viscous and flows more slowly from the tip to the substrate. Controlling humidity and tip contact time (or scan speed) therefore allows precise control the thickness of the lipid multilayer structures at a scale between 1 and 100 nm (FIG. 3).

FIG. 2 shows an array 212 of lipid multilayer nanostructure 214 on a substrate 216.

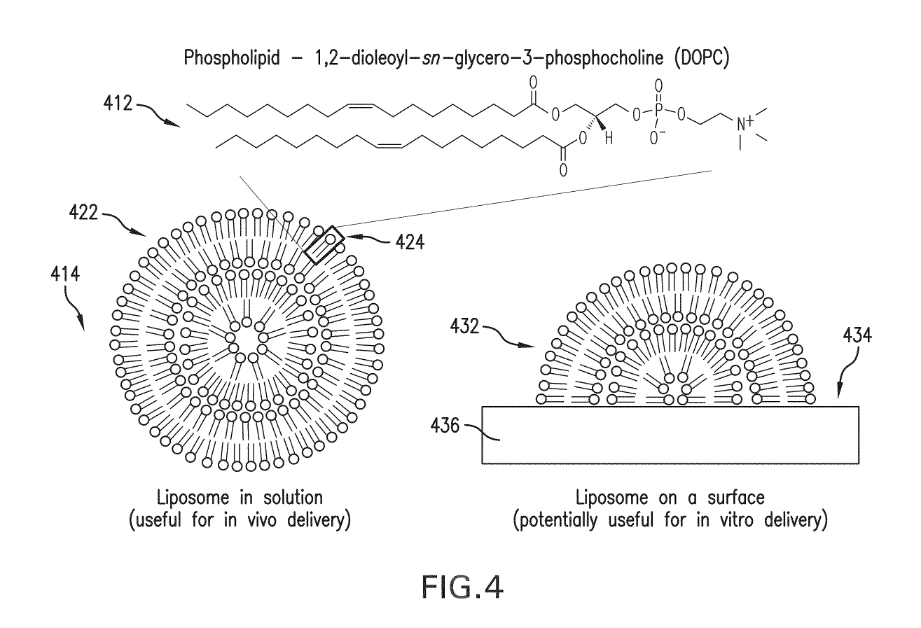

Nanoscience seeks to determine how a material's properties change as its size changes between the molecular and the macroscopic scale. Biology can be viewed as a kind of natural nanotechnology that provides a proof of concept and inspiration for nanoscientists.⁵⁹ Biomolecular nanoscience that uses biological molecules (e.g., DNA and proteins) interfaced with synthetic, inorganic materials is a well-established and productive field.^{60,61,62,63,64,65} Lipid-based nanoscience arguably exists in the form of the colloid chemistry and nano-emulsions such as liposomes, which are typically solution based. In water, DOPC spontaneously self-organizes to form liposomes with lamellar bilayer structures, such as the one sketched in FIG. 4. FIG. 4 shows chemical and supramolecular structures of liposomes and surface-supported lipid nanostructures. The chemical structure of 1,2-dioleoyl-sn-glycero-3-phosphocholine (DOPC), a typical phospholipid that may be used in lipid microstructure of the present invention, is indicated by arrow 412. FIG. 4 also shows example of one type of liposome supramolecular structure that self-assembles in water 414, i.e., multilamellar liposome 422 that is comprised of DOPC, as indicated by box 424. FIG. 4 also shows a surface-supported lipid multilayer liposome 432 on a surface 434 of a substrate 436. FIG. 4 shows one possible supramolecular structure and serves the purpose of comparing the structure of liposomes in solution with surface-supported liposomes or lipid multilayer nanostructures. The present invention combines the advantages of multilayered liposomes with those of surface-supported lipid bilayers in a microarray format capable of taking advantage of novel optical properties that emerge from lipid nanostructures.

In one embodiment of the present invention, the lipid chemical functions and the dynamic nanostructural properties of lipid-multilayer microarray and nanoarrays may be integrated in a format that provides highly integrated multimaterial sensors. Such arrays maybe fabricated by DPN and lipid multilayer stamping. Such arrays may be made out of multiple materials and may be made using high throughput techniques. Descriptions of the formation, properties and various applications of lipid multilayer nanostructures have been described.^{1,20,21,66,67,68,69}

Lipid multilayer microstructures have been shown to be suitable for multimaterial DPN allowing control of multilayer thickness.²¹ Lipid multilayers microstructures have been used for multiplexed lipid DPN for protein templating and cell culture.²⁰ Lipid multilayers microstructures have been made using DPN under water.⁶⁹ Lipid multilayer technology has been used to fabricate optically active nanostructures capable of biological sensing.¹ High-throughput optical quality control of lipid nanostructures has been demonstrate.⁶⁸

In one embodiment the present invention provides a method in which an array of lipid multilayer gratings is exposed to a fluid containing one or more analytes. Each of the lipid multilayer gratings comprises iridescent lipid multilayer nanostructures. After being exposed to the fluid, one or more incident lights are shone on the array and the light scattered by the gratings is detected by a detector. Based on the scattered light detected by the detector, the presence and/or concentration of one or more analytes the fluid is determined. In one embodiment of the present invention, the presence and/or concentration of the one or analytes may be determined by comparing the scattered light detected to a standard reading for the detector for the array being exposed to a control fluid. In one embodiment of the present invention, the presence and/or concentration of the one or analytes may be determined by comparing the scattered light detected to a reading for the detector for the array being exposed to a control fluid.

In one embodiment of the present invention, two or more of the gratings may comprise different lipids.

In one embodiment of the present invention, lipid multilayer gratings may comprise one or more phospholipids.

In one embodiment of the present invention, the fluid to which the array is exposed may be a liquid such as water.

In one embodiment of the present invention, the fluid to which the array is exposed may be a gas such as air.

### EXAMPLES

### Example 1

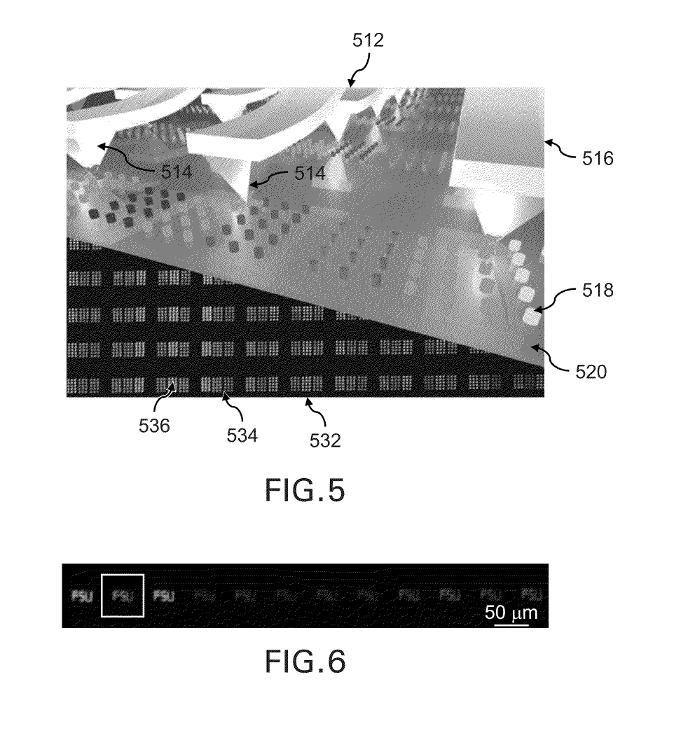

A variety of chemical functions may be readily integrated onto the same surface with high resolution and registry using parallel and multiplexed lipid DPN (see FIGS. 5, 6, 7, 8, 9, 10, 11).²⁰ Parallel DPN is patterning with multiple tips simultaneously in an array, and multiplexed DPN is the transfer of different inks from the different tips in the array. In this example, DOPC ink is used as a carrier for other functional materials, such as fluorescently labeled lipids (FIG. 5).⁶⁸ FIG. 5 shows massively parallel and multiplexed DPN. Shown in a top portion 512 of FIG. 5 is a schematic drawing of different tips 514 in a parallel array 516 integrating different inks 518 on a surface 520. Shown in a bottom portion 532 of FIG. 5 is a fluorescence micrograph of phospholipid patterns 534 of dots 536 with a neighboring dot spacing of 2 microns.^{4,6,9}

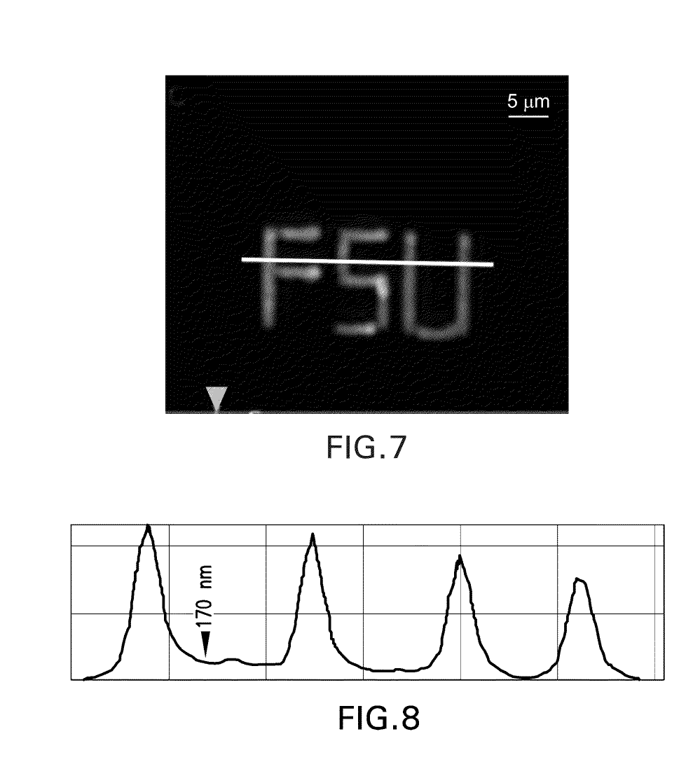

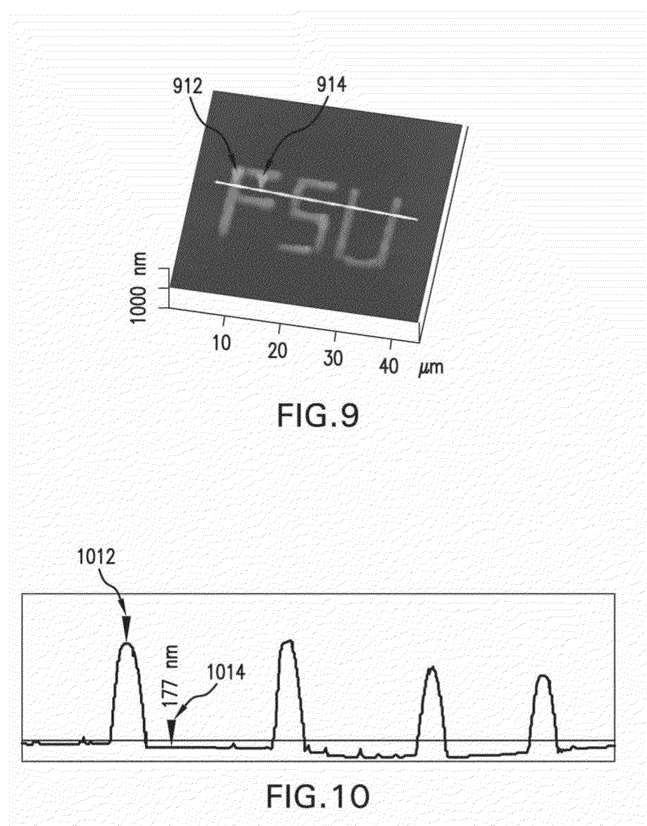

In order to test this approach of quantifying the feature height by using the florescence intensity of lipid features, the fluorescence intensity is used to measure the height of a “FSU” pattern created with lines as shown in FIGS. 6, 7, 8, 9 and 10. The calibration curve used to measure the height of the FSU letters has a slope of 0.337 grey values/s/nm (not shown). The FSU pattern is created and imaged under the fluorescent microscope using the 10× objective lens over different exposure times (800 μs-8 s), and its height is immediately measured with tapping mode AFM. FIG. 6 is a fluorescent microscope image of a large area (0.12 mm²) FSU pattern created by moving the DOPC-coated tip at a tip speed of 75 nm/s. FIG. 7 shows a close-up fluorescent microscope image of the FSU pattern imaged at 2 seconds exposure time. The height of the same “F” letter measured across the region denoted by the white line in FIG. 8 using the calibration curve of FIG. 9, is estimated to be ˜170 nm using Equation 1 below:

$$\begin{matrix}Height ( nm ) = \frac{\begin{matrix}Measured intensity from \\ fluorescence image ( grey values )\end{matrix} }{\begin{matrix}Slope of the line calibration curve \\ ( grey values \text{/} s \text{/} nm ) \times Exposure time ( s )\end{matrix} } \\ = \frac{115}{0.337 \times 2} \\ = 170\end{matrix}$$

FIG. 9 is AFM height image of the same FSU logo of FIG. 7 at the same exposure time, i.e., 2 seconds, with a measurement performed at the same location of the white line as shown in FIG. 10. The height is measured to be 177 nm as shown in FIG. 10. The error between the estimated feature height obtained using Equation 1 and the measured height is within an error of ˜4%. FIG. 9 includes two reference marks 912 and 914 that correspond to reference marks 1012 and 1014, respectively in FIG. 10.

The actual feature heights of ten different measurements measured by AFM are compared to those estimated using the fluorescence intensity of the structures, and the differences are found to be within an average of 7%±4% of the feature heights measured with AFM. Further, the lowest height of the fluorescent microstructure that could be reproducibly quantified by this approach is ˜10 nm, which is the equivalent of three DOPC lipid bilayers (which are 3.5 nm).

This close matching of the estimated feature height (from calibration curves obtained using fluorescence intensity measurements) to the actual feature height obtained using AFM measurements in a different experiment validates this approach of using optical quality control to determine feature height. This control over height may be important in developing novel applications of lipid microstructures as diffraction gratings. Further, this nonintrusive optical approach may be extended to systems where the lipid microstructures can be envisioned to act as carriers of other biomaterials essential to understanding cell-structure relationships. With the base lipid feature height vs. intensity calibrated, it may be possible to estimate the amount of biomaterial carried with the lipid microstructure. This approach may also be used with other similar liquid (lyotropic) biocompatible ink systems using optical quality control as the height-determining method. Optical quality can be especially useful for large-area feature height determination where slow AFM scanning is not desirable.

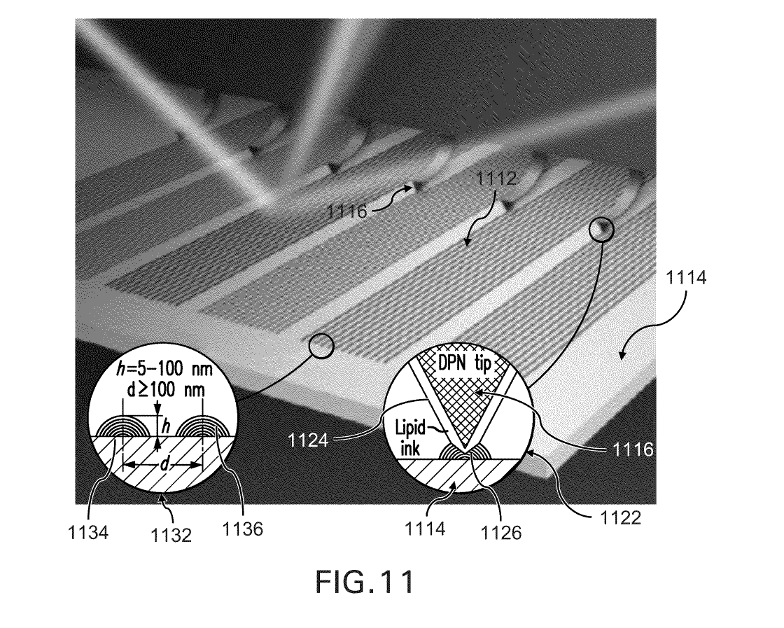

A unique aspect of lipid DPN is the ability to control multilayer thickness, and the inventor recently took advantage of this capability to produce lipid multilayer gratings, which are optical-diffraction gratings composed of lipid, as illustrated in FIG. 11.¹ Parallel and multiplexed DPN was used to deposit multiple lipids simultaneously with controllable multilayer heights, laterally structured to form arbitrary patterns (e.g., diffraction gratings) with feature sizes on the same scale as UV, visible, or infrared light. In situ observation of the light diffracted from the patterns can be carried out during DPN and used for optical quality control without the need for fluorescent labels.

When a diffraction grating is illuminated with white light from an angle, the color of the light diffracted from the structures is described by the grating equation:

d(sin θₘ+sin θᵢ)=mλ (2)

where d is the period of the grating, θₘ and θᵢ are the angles of diffraction maxima and incidence, respectively, m is the diffraction order, and λ is the wavelength of light. Although the color of light observed depends only on the angles θₘ and θᵢ, the intensity or efficiency of light diffracted from the gratings depends both on the quality and, especially, on the height of the grating.

FIG. 11 shows lipid multilayer diffraction gratings 1112 deposited on a substrate 1114 using DPN tips 1116. An inset 1122 shows a DPN tip 1116 and lipid ink 1124 being deposited as a line 1126 of a diffraction grating 1112. Inset 1132 shows two lines, i.e., lines 1134 and 1136, of a diffraction grating 1112.

As shown in FIG. 11, parallel DPN tip arrays are used to deposit multiple lipids simultaneously with controllable multilayer heights, laterally structured to form arbitrary patterns such as diffraction gratings with feature sizes on the same scale as UV, visible or infrared light. In situ observation of the light diffracted from the patterns may be carried out during DPN and used for high-throughput optical quality control without the need for fluorescence labels.

The ability of lipid DPN to control the lipid multilayer height constructively is important to forming multilayer structures. With the exception of capillary assembly, the majority of lipid patterning methods are limited to single monolayers or surface-supported lipid bilayers.

### Example 2

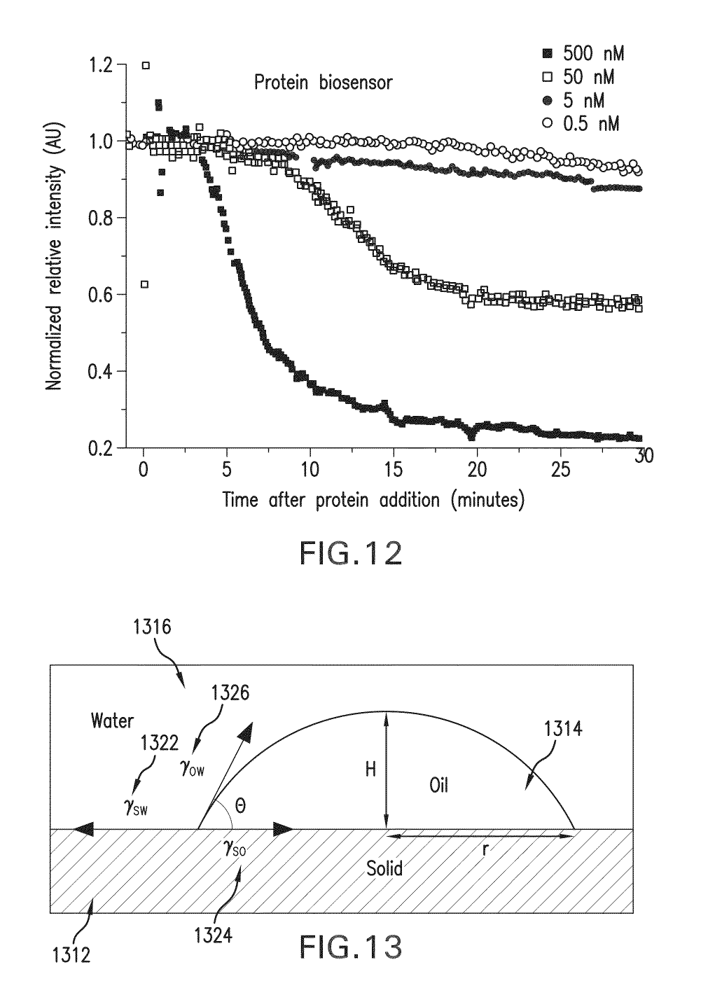

Although diffraction gratings are one of the simplest and best-studied photonic structures, lipid multilayer gratings are a fundamentally new type of material, because they are fluid, innately biocompatible, and immersible in water. Incorporation of functional materials such as biotinylated lipids into the gratings allows them to be used as label-free biosensors when the intensity of diffracted light is monitored as a function of time during protein binding, as shown in FIG. 12. Monitoring the intensity of light diffracted from lipid multilayer gratings on exposure to analytes permits optical detection of protein binding without any fluorescent labels. For example, FIG. 12 shows the optical response of biotinylated gratings upon exposure to streptavidin protein at different concentrations. The decrease in intensity is due to the dewetting mechanism, which results in a lower diffraction efficiency. The observed limit of detection of 5 nM after 15 min is comparable to that of solid grating-based sensors, which are typically diffusion limited at concentrations on the order of ˜5 nM, but after incubation for 90 min, it is possible to observe significant dewetting of biotinylated gratings, as compared to the pure DOPC control gratings, at a protein concentration of 500 pM. As the dewetting detection mechanism depends on a change in surface energy, the sensitivity for a particular analyte may be optimized by adjustment of the sensitivity of the membrane tension to ligand binding, as is the case in many cell-signaling processes and model membrane systems as described in Chiu, D. T. et al., “Chemical transformations in individual ultrasmall biomimetic containers,” Science 283, 1892-95 (1999), the entire contents and disclosure of which is incorporated herein by reference. Furthermore, phospholipid bilayers are highly resistant to nonspecific protein binding, and it is therefore possible to carry out the same detection of protein added to fetal calf serum. The response of the grating to protein binding depends on the grating height; higher gratings give the best response for protein detection at low concentration. Therefore, observing a quantitative concentration-dependent response requires use gratings of equivalent height (35+5 nm as determined by diffraction intensity calibration) for the experiment series shown in FIG. 12.

The sensing mechanism can be understood in terms of physical adhesion (FIG. 13) among solid 1312, oil 1314 and water 1316, where γ_{sw} 1322, γₛₒ 1324, γ_{ow} 1326 are the interfacial energies of the solid-water, solid-oil, and oil-water interfaces, respectively. A change in any of these interfacial energies results in a change in the lipid multilayer grating height, which can be detected optically.

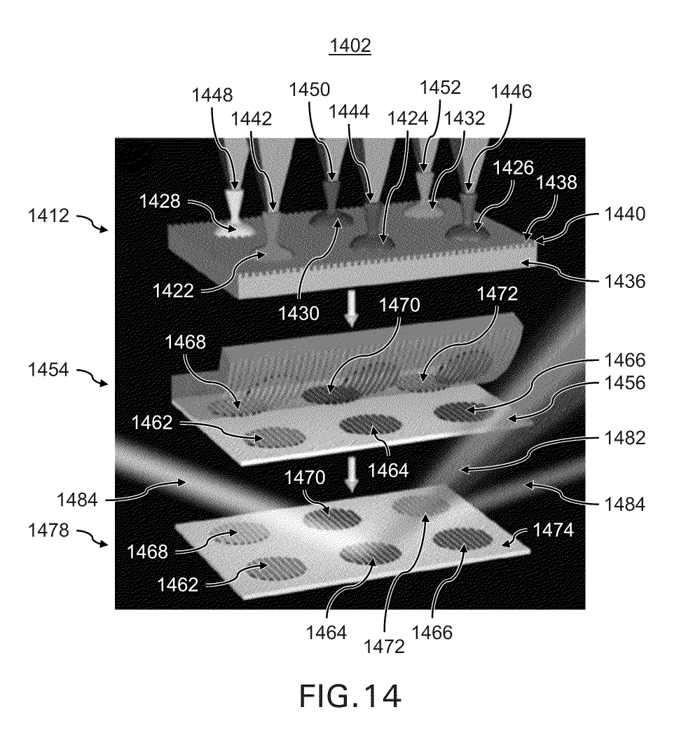

DPN although excellent for rapid prototyping purposes, has been limited as a manufacturing tool because of associated cost, inhomogeneities in thickness between different tips in massively parallel arrays, and limits on the types of lipid inks that can be used under ambient conditions. In order to produce a sufficient quantity of lipid multilayer gratings formed from multiple materials in a manner suitable for sensor array testing and development, in one embodiment the present invention employs scalable process for lipid multilayer grating fabrication, as shown in FIGS. 14, 15, 16, 17, and 18. This fabrication method combines the lateral patterning capabilities and scalability of microcontact printing with the topographical control of nanoimprint lithography and the multimaterial integration aspects of dip-pen nanolithography in order to create nanostructured lipid multilayer arrays.

FIG. 14 shows a multilayer stamping process 1402 of the present invention used in this example. In step 1412 lipid inks 1422, 1424, 1426, 1428, 1430 and 1432 are spotted on a topographically structured stamp 1436 using respective dip pens 1442, 1444, 1446, 1448, 1450 and 1452 using DPN. Topographically structured stamp 1436 has grooves 1438 and ridges 1440. In step 1454, inked topographically structured stamp 1436 prints lipid inks 1422, 1424, 1426, 1428, 1430 and 1432 on a substrate 1456 as respective stamped spots 1462, 1464, 1466, 1468, 1470 and 1472. Stamped spots 1462, 1464, 1466, 1468, 1470 and 1472 together form a patterned array 1474. Stamped spots 1462, 1464, 1466, 1468, 1470 and 1472 are each a diffraction grating. Step 1478 shows spot 1464 scattering a scattered portion 1482 of white light 1484.

Stamping techniques that may be used to formed patterned arrays of the present invention are also described and shown in U.S. Patent Application No. 2012/02582892 to Lenhert et al., entitled “METHODS AND APPARATUS FOR LIPID MULTILAYER PATTERNING,” published Oct. 11, 2012, the entire contents and disclosure of which are incorporated herein by reference. Other stamping techniques that may be used to form patterned arrays of the present invention are described and shown in International Patent Application No. PCT/IB2013/055762 to Lenhert et al., entitled “SCALABLE LIPOSOME MICROARRAY SCREENING, filed Jul. 12, 2013, the entire contents and disclosure of which are incorporated herein by reference.

### Example 3

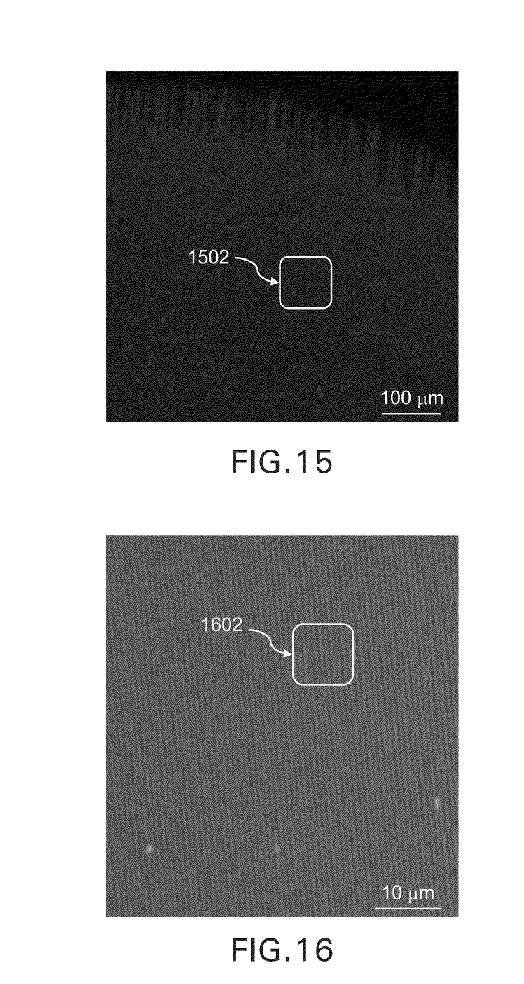

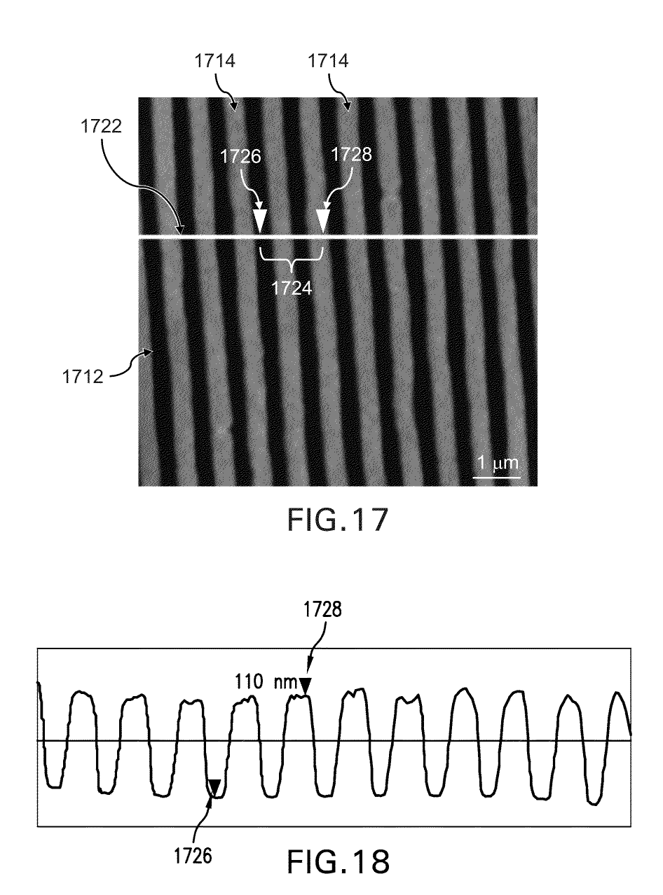

Lipid multilayer gratings formed with the gel-phase lipid DPPC. Gratings may also be created with other lipids besides fluid DOPC. FIGS. 15, 16 and 17 show green diffraction together with the corresponding AFM image of the stamped grating structures of a gel-phase phospholipid like 1,2-dipalmitoyl-sn-glycero-3-phosphocholine (DPPC), which cannot be patterned by DPN at room temperature, as it is not fluid at room temperature (Tm=41° C.). The gratings gave three distinct diffraction colors (red, green, and blue) at different angles of incident light. Importantly, these DPPC gratings can be immersed in water under ambient conditions (humidity up to 60%), which is a significant practical advantage over DOPC-based lipid multilayer gratings which require that immersion in water be carried out in a dehydrating atmosphere, such as pure nitrogen.⁸⁵ This technique may also be used to create diffraction gratings with lipids that are not phospholipids, in particular those that cannot be patterned by DPN or techniques based on spin-coating multilayers.³³ The steroid cholesterol was used for this purpose, as it is a fundamentally different type of biological lipid yet is still an integral component of animal cell membranes. FIG. 15 is red diffraction obtained from stamped DPPC gratings with a 140 nm tall and 700 nm pitch stamp. Region 1502 is enclosed in a white square. FIG. 16 is an optical micrograph with surface-enhanced ellipsometric contrast (SEEC) imaging of a region 1502 in FIG. 15 showing DPPC grating lines over a large area. Region 1602 is enclosed in a white square. FIG. 17 is an AFM height image of a region 1602 in FIG. 16. FIG. 17 as grating 1712 that comprises an array of linear lipid multilayer nanostructures 1714. A white line 1722 extends across grating 1712. A portion 1724 of white line 1722 extends between triangle s 1726 and 1728 FIG. 18 is a line trace along line 1702 in FIG. 17 showing an average height of 110 nm±10 nm. The DPPC gratings are stamped onto a commercially available silicon oxide surface (Surf) for greater optical contrast.

### Example 4

### Fabrication and Characterization of Iridescent Arrays Composed of Multiple Lipids

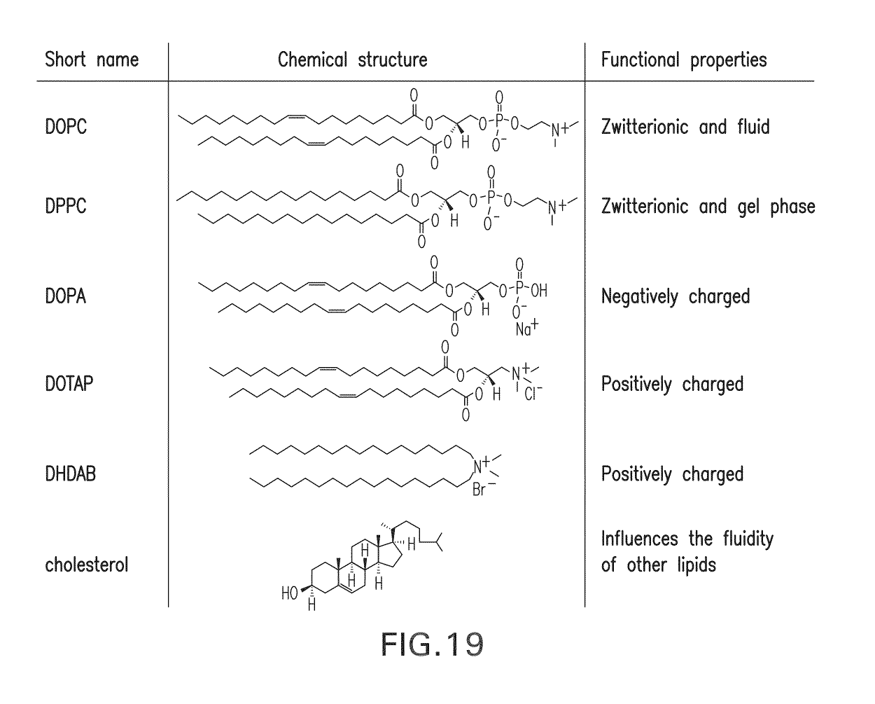

In one embodiment the present invention provides a photonic nose that employs arrays of sensor elements that differ in chemical functionality and/or physical properties. Examples of the types of lipids that may be used in a photonic nose of the present invention are shown in Table 1 of FIG. 19. Table 1 lists the acronyms, chemical structures and functional properties the lipids integrated in the multicomponent lipid arrays of this example.

Both DPN and lipid multilayer stamping may be carried out using these lipids systematically mixed together in different ratios in order to determine which lipid mixtures are compatible with the fabrication methods.

The lipids listed in Table 1 of FIG. 19 vary by fluidity and charge of the headgroup. The effect of these parameters on the quality of the gratings will be determined. Characterization is carried out using fluorescence microscopy, AFM, and measurement of iridescence. The mixtures are doped with fluorescently labeled lipids enabling rapid and initial, although low lateral resolution characterization by fluorescence microscopy.⁶⁸ AFM is used to characterize feature uniformity and shape at high resolution. While AFM has traditionally required a significant amount of patience and skill to carry out, new software on this system (called ScanAssyst) enables automated parameter optimization which makes it possible for undergraduates to quickly obtain quality and rewarding images. Most importantly, the iridescence of these surfaces is characterized, as this is the property that allows these surfaces to be used as optical sensors. A simple method employing an optical microscope and a white light source are used to characterize iridescence.⁸⁴

### Example 5

### Determination of the Relationship Between Lipid Composition, Optical Properties, and Environmental Conditions such as pH, Salt Concentration, and Temperature

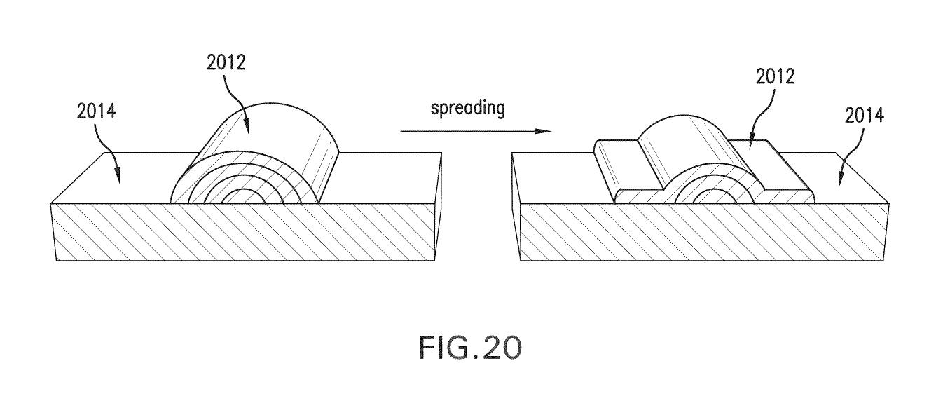

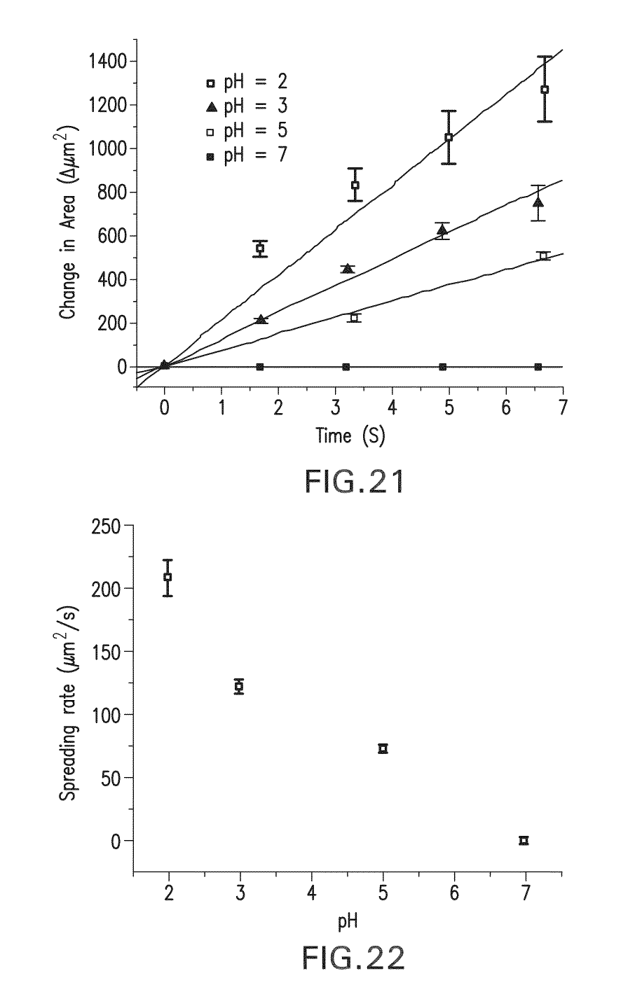

In order to understand the environmental factors that influence the color change of the sensor elements, sensor elements are characterized in different solution conditions, specifically by varying pH, salt concentration and temperature. These parameters are varied while observing the samples using the characterization methods described above. In the experiment of this example, the lipid DOPC is observed to spread on plasma cleaned glass using fluorescence microscopy, and a strong dependence on pH of the solution is observed. AFM on these samples is carried out in liquid in order to directly observe nanoscale changes in shape of the grating lines in response to changes in pH and salt concentration. Other shape changes, such as intercalation of salts and dewetting are also expected by different lipid mixtures,¹ and identifying these different responses will allow design of optimal lipids for inclusion in the photonic nose. Additionally, Differential Scanning calorimetry (DSC) is carried out on lipid mixtures of interest in order to determine their temperature dependent phase transition behavior. The structuring of lipids into photonic structures provides a label-free method of observing dynamic structural changes in the lipid multilayer morphologies. These changes may be understood in terms of liquid adhesion to a solid surface where the lipid multilayers are, essentially, structured microscopic and nanoscopic oil droplets adherent on a surface. Three examples of shape changes are spreading, dewetting and intercalation of materials into the multilayer structure, as schematically illustrated in FIG. 20. FIG. 20 shows spreading of lipid layers 2012 on substrate 2014. Results from such an experiment that has are shown in FIGS. 21 and 22. Data showing the change in area spreading of lipids as a function different time, measured at different pH values is shown in FIG. 21. FIG. 22 shows a plot of the spreading rate (slopes of the lines in FIG. 21).

### Example 6

### Signal Amplification by Means of Confined Chemical Reactions

Lipid multilayer gratings are useful biomimetic biosensors in that, like living cells, they provide a compartment in which chemical reactions can be confined and used to amplify signals. In order to take advantage of this possibility, a simple organic reaction is used. The reaction is a Grubbs ring-opening metathesis polymerization (ROMP) reaction of norbornene derivatives, specifically dicyclopentadiene (DCPD), which a lipophilic molecule that can be encapsulated in lipid multilayer gratings (data not shown). This living polymerization reaction features high catalyst turnover (for signal amplification), and polymerization within the fluid layer is expected to produce the shape changes necessary for changing the optical properties of the grating. Assuming large signal amplification and multiple gratings, it will be possible to calculate precise levels of analyte (Grubbs catalyst) even at ultra-low concentrations. The ability of Grubbs-type catalysts to convert liquid dicyclopentadiene monomers into a solid cross-linked polymer has been exploited in many materials applications, including self-healing.³ By encapsulating dicyclopentadiene in the lipid multilayer gratings, and exposing the sensor elements to Grubbs catalyst dissolved in aqueous solution while monitoring diffraction it is possible to optically detect the presence of the catalyst in the solution. An optical microscope and a white light source are used to characterize iridescence of the samples.⁸⁴ AFM on these samples is carried out in liquid in order to directly observe nanoscale changes in shape of the grating lines upon exposure to the catalyst.

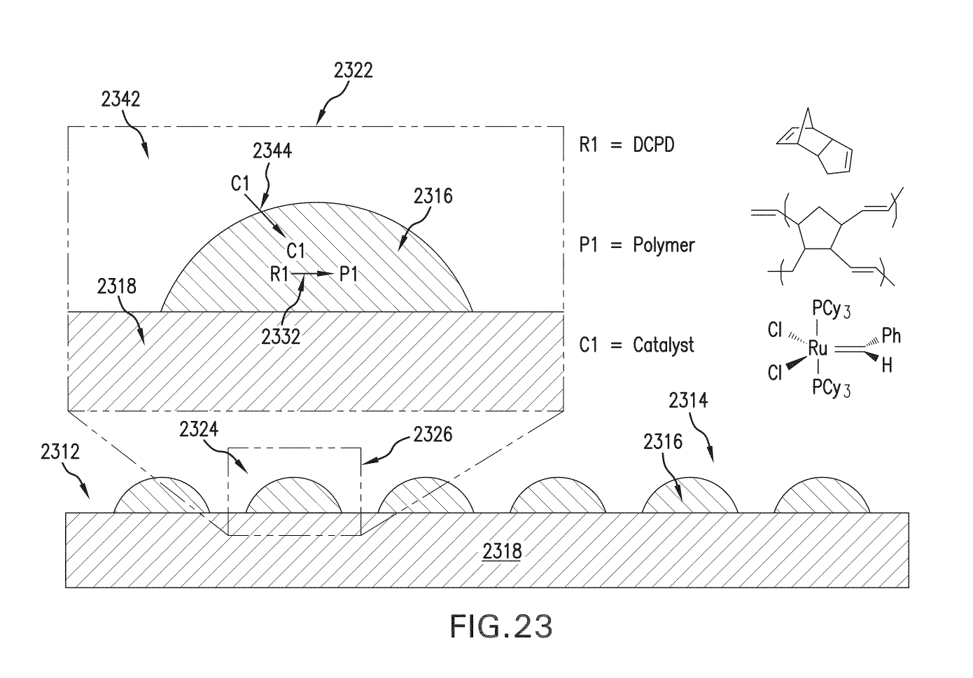

FIG. 23 is a schematic illustration of a process according to one embodiment of the present invention that may be used to detect the presence of a catalyst, such as a Grubbs catalyst, in an aqueous solution. FIG. 23 shows a patterned substrate 2312 comprising an array 2314 of lipid multilayer grating lines 2316 on a substrate 2318. An inset 2322 shows an enlarged version of portion 2324 of patterned substrate 2312 enclosed in a dashed box 2326. A reagent R1 (DCPD) is placed in each lipid multilayer grating line 2316. A catalyst C1 catalyzes the reaction from reagent R1 to a product P1 (a polymer) as indicated by arrow 2332. Catalyst C1 is dissolved or dispersed by means of emulsification in an aqueous solution 2342 and diffuses into each lipid multilayer grating line 2316 as shown by arrow 2344. The presence of product P1 in lipid multilayer grating lines 2316 has an effect on the light scattering properties of lipid multilayer grating lines 2316. This change in light scattering properties can be detected using a detector to detect the presence and/or concentration of catalyst C1 in aqueous solution 2342.

Although only one reagent is shown in FIG. 23, in some embodiments of the present invention there may be two or more reagents whose reaction is catalyzed by a catalyst.

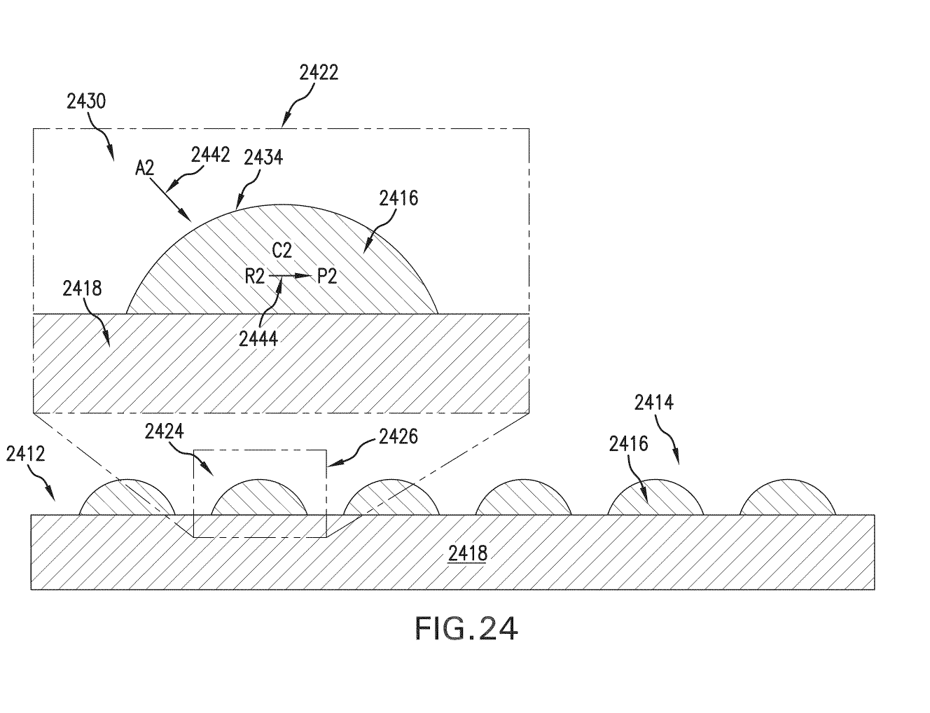

FIG. 24 is a schematic illustration of a signal amplification process according to one embodiment of the present invention. FIG. 24 shows a patterned substrate 2412 comprising an array 2414 of lipid multilayer grating lines 2416 on a substrate 2418. An inset 2422 shows an enlarged version of portion 2424 of patterned substrate 2412 enclosed in a dashed box 2426. Lipid multilayer grating lines 2416 are immersed in an aqueous solution 2430 containing an analyte A2. Encapsulated in each lipid multilayer grating line 2416 is a reagent R2 and a catalyst C2 that catalyzes a reaction, indicated by arrow 2432. Analyte A2 from aqueous solution 2430 interacts with catalyst C2 either on an exterior surface 2434 of each lipid multilayer grating line 2416 or within lipid multilayer grating line 2416 as shown by arrow 2442. In some embodiments of the present invention, analyte A2 may diffuse into lipid multilayer grating line 2416 to interact with catalyst C2. As a result of the interaction between analyte A2 and catalyst C2, catalyst C2 catalyzes a reaction, indicated by arrow 2444 that causes R2 to produce product P2. The presence of product P2 in lipid multilayer grating lines 2416 has an effect on the light scattering properties of lipid multilayer grating lines 2416. This change in light scattering properties can be detected using a detector to detect the presence and/or concentration of analyte A2 in aqueous solution 2430.

Depending on the analyte, reagent and catalyst the product of the reaction may be various different types of chemical or biochemical products. In some embodiments of the present invention there may be two or more reagents. In some embodiments of the present invention, there may two or more products produced from the reaction that is catalyzed by the catalyst.

In some embodiments of the present invention, instead of reacting with the catalyst on the surface of the lipid multilayer grating line as shown in FIG. 24, the analyte may diffuse into the lipid multilayer grating line where the catalyst catalyzes the reaction of the analyte with the reagent to form the product.

An example of a type of amplification process that is illustrated in simplified form in FIG. 24 is an enzyme-linked immunosorbent assay (ELISA). For this purpose, one antibody is linked to the lipid multilayer grating lines, and a second, enzyme-linked antibody (a “catalyst”) will be preincubated with the analyte. The enzyme substrates are encapsulated within the lipid multilayer grating lines so that, when the analyte binds to one of the lipid multilayer grating lines, a reaction is catalyzed within the grating to induce a shape change. For example, an enzyme-linked antibody with phospholipase activity could be used to catalyze the cleavage of phospholipid only in the presence of an antigen. For example, phospholipase C (the “catalyst”) could be used to cleave the phospholipid phosphatidylinositol (the “reagent”) into inositol triphosphate (IP₃) and diacylglycerol (DAG) (the “products”). This approach may permit single-molecule detection even if the chemical reaction used to amplify the signal is four orders of magnitude less efficient than ELISA. (The basis of this estimate is that a single molecule in one lipid droplet of 50×500×5000 nm is at a concentration of ˜10⁻⁸ M, and ELISA is sensitive down to concentrations of ˜10⁻¹² M.).

In one embodiment of the present invention that is also illustrated by FIG. 24, the analyte may activate a catalyst that is already present in the lipid multilayer. As shown in FIG. 24, when a lipid multilayer grating line 2416 is exposed to analyte (A2), catalyst (C2) become active and catalyzes the reaction of converting R2 to P2. An example of such a catalyst may be a G-protein coupled receptor, which activates a G-protein only in the presence of the analyte.

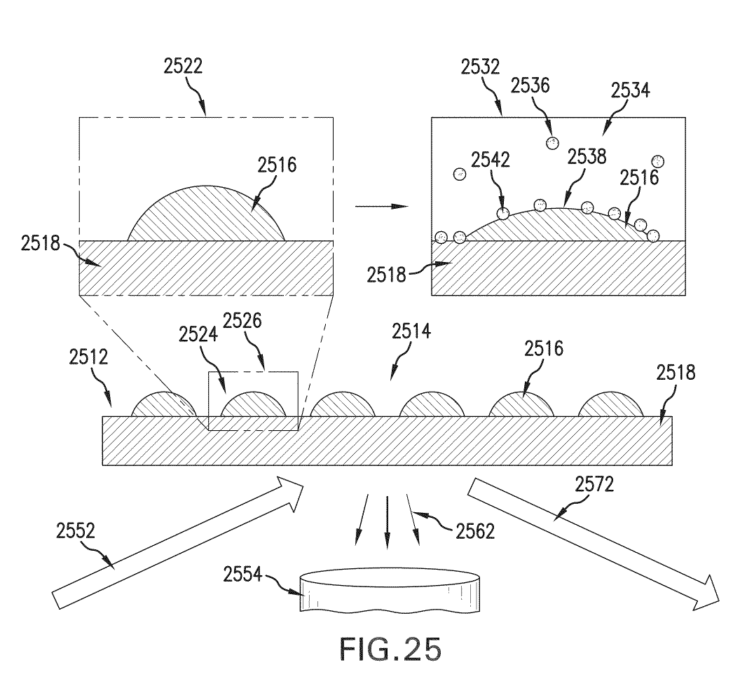

FIG. 25 is a schematic illustration of an analyte detection process according to one embodiment of the present invention. FIG. 25 shows a patterned substrate 2512 comprising an array 2514 of lipid multilayer grating lines 2516 on a transparent or translucent substrate 2518. In one embodiment, substrate 2518 may be glass or a plastic. Inset 2522 shows an enlarged version of portion 2524 of patterned substrate 2512 enclosed in a dashed box 2526. Inset 2532 shows a lipid multilayer grating line 2516 immersed in an aqueous solution 2534 containing an analyte 2536. At least some of analyte 2536 binds to lipid multilayer grating line 2516 to form a bound analyte 2542 to thereby cause lipid multilayer grating line 2516 to spread as shown in FIG. 25. In one embodiment of the present invention, if lipid multilayer grating line 2516 contains a reagent and a catalyst, the binding of analyte may bind to a catalyst (not shown) on an exterior surface 2538 of lipid multilayer grating line 2516. As a result of the interaction between analyte 2536 and the catalyst, the catalyst catalyzes a reaction of the reagent that produces a product that causes lipid multilayer grating line 2516 to spread. This change in shape of lipid multilayer grating line 2516 changes the light scattering properties of lipid multilayer grating line 2516. This change in light scattering properties can be detected using an incident white light 2552 and a detector 2554. A portion of incident white light 2552 travels through substrate 2518 and is scattered by lipid multilayer grating lines 2516 as scattered light 2562 (shown as three diverging arrows). By comparing scattered light 2562 detected by detector 2554 for patterned substrate 2512 before and after patterned substrate is immersed in aqueous solution 2534 containing analyte 2536, it is possible to detect the presence and/or concentration of analyte 2536 in aqueous solution 2534. A portion of incident white light 2552 is reflected by substrate 2518 as reflected slight 2572.

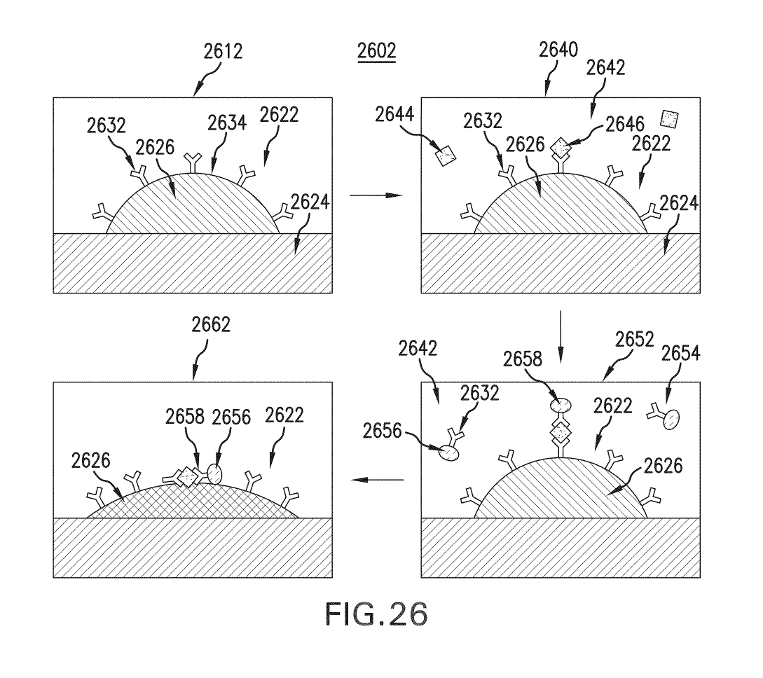

FIG. 26 is a schematic illustration of enzyme-linked immunosorbent assay (ELISA) process 2602 according to one embodiment of the present invention. Box 2612 shows a patterned substrate 2622. Patterned substrate 2622 comprises a transparent or translucent substrate 2624 and an array of lipid multilayer grating lines 2626. For illustration purposes, only one lipid multilayer grating line 2626 is shown in FIG. 26. Box 2612 shows an antibody 2632 that is immobilized on an exterior surface 2634 of lipid multilayer grating line 2626. Box 2640 shows patterned substrate 2622 immersed in an aqueous solution 2642 containing an analyte 2644 that binds to antibody 2632 immobilized on exterior surface 2634 of lipid multilayer grating line 2626 to thereby form a bound analyte 2646. Box 2652 shows an enzyme-linked antibody complex 2654 being added to aqueous solution 2642. Antibody complex 2654 comprises an enzyme 2656 linked to antibody 2632. A portion of antibody complex 2654 added to aqueous solution 2642 binds to bound analyte 2646 to thereby form a bound enzyme-linked antibody complex 2658. Box 2662 shows enzyme 2656 catalyzing a reaction in lipid multilayer grating line 2626 that alters the shape of lipid multilayer grating line 2626. In box 2652 this change in shape is shown as a spreading of lipid multilayer grating line 2626, but other changes in shapes of lipid multilayer grating line 2626 are possible. The change in shape of lipid multilayer grating line 2626 changes the light scattering properties of lipid multilayer grating line 2626.

The change in the light scattering properties of the lipid multilayer grating lines of the patterned substrate due to the binding of the analyte to the immobilized antibody on the lipid multilayer grating lines and the subsequent binding of the enzyme-linked antibody to the analyte can be detected using an incident white light and a detector (not shown in FIG. 26) in a fashion similar to what is shown in FIG. 25. According to one embodiment of the present invention, a portion of incident white light may travel through substrate and be scattered by the lipid multilayer grating lines as scattered light. By comparing the scattered light detected by detector for the patterned substrate before and after the ELISA assay is carried out using patterned substrate, it is possible to detect the presence and/or concentration of the analyte in the aqueous solution.

### Example 7

### Assay Employing Ion Channel in a Lipid Multilayer Structure

In one embodiment of the present invention, the present invention provides an assay employing an array of lipid multilayer structures with ion channels. An analyte that is to be detected may activate the ion channels of the lipid multilayer structures. Once activated, the ion channels allow materials to enter and/or exit a lipid multilayer structure. For example, incorporation of a ligand gated ion channel in the lipid multilayer would allow ions to enter or exit the lipid multilayer only in the presence of the analyte. Each ion channel of a lipid multilayer structure mimics the behavior of an ion channel, such as a calcium channel, in a cell membrane.

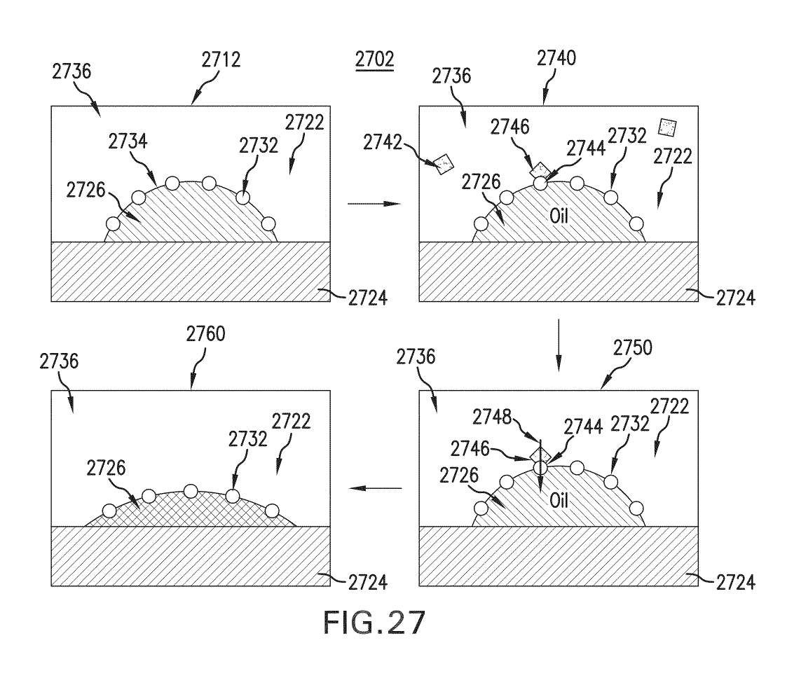

FIG. 27 is a schematic illustration of an assay process 2702 according to one embodiment of the present invention in which an analyte activates an ion channel in a lipid multilayer structure. Box 2712 shows a patterned substrate. Patterned substrate 2722 comprises a transparent or translucent substrate 2724 and an array of lipid multilayer grating lines 2726. For illustration purposes, only one lipid multilayer grating line 2726 is shown in FIG. 27. Box 2712 shows ion channels 2732 on an exterior surface 2734 of lipid multilayer grating line 2726. Patterned substrate is immersed in water 2736. Box 2740 shows analyte 2742 added to water 2736 to thereby form an aqueous solution. One analyte 2742 is shown binding to an ion channel 2744 of ion channels 2732 to form a bound analyte 2746 that activates ion channel 2744, i.e., causes ion channel 2744 to open as indicated by arrow 2748. Box 2750 shows bound analyte 2746 causing ion channel 2744 to open. The opening of ion channel 2744 allows ion diffusion into lipid multilayer grating line 2726 which causes lipid multilayer grating line 2726 to change its shape. Box 2760 s this change in shape is shown as a spreading of lipid multilayer grating line 2726, but other changes in shapes of lipid multilayer grating line 2726 are possible. The change in shape of lipid multilayer grating line 2726 changes the light scattering properties of lipid multilayer grating line 2726.

The change in the light scattering properties of the lipid multilayer grating lines of the patterned substrate due to the binding of the analyte to the ion channels on the lipid multilayer grating lines and the subsequent infusion of ions into the lipid multilayer grating lines can be detected using an incident white light and a detector (not shown in FIG. 27) in a fashion similar to what is shown in FIG. 25. According to one embodiment of the present invention, a portion of incident white light may travel through substrate and be scattered by the lipid multilayer grating lines as scattered light. By comparing the scattered light detected by detector for the patterned substrate before and after the assay is carried out using patterned substrate, it is possible to detect the presence and/or concentration of the analyte in the aqueous solution.

Examples of analytes that may gate, or open and close an ion channels as shown in the assay of FIG. 27 include voltage gated ion channels such as calcium ions (Ca²⁺), sodium channels (Na⁺), potassium channels (K⁺), or ligand gated ion channels such as neurotransmitters, or agonist drugs for acetylcholine receptors, glutamate-gated receptors, γ-aminobutyric acid-gated GABA receptors.

### Example 7

### Electrostatic Interactions with Analyte Change Shape of Lipid Multilayer Structure

In another embodiment, the analyte may interact with the surface of a lipid multilayer by electrostatic interactions, thus changing the interfacial tensions of the lipid multilayer structure and thus the shape of the lipid multilayer structure.

### Example 8

### Determination of Water Quality Using the Concept of a Photonic Nose

A photonic nose comprising iridescent lipid nanostructure arrays composed of various different lipid mixtures are made. Lipid mixtures found to have different responses to changes in pH, salt concentration, and temperature are selected. Addition of metal salts of Cu, Ni, Fe, Cr that are relevant to wastewater treatment are added to the aqueous solution and the optical response of the photonic nose are measured using a CCD camera. Upon successful detection, the different metals are mixed together and the ability for the photonic nose to distinguish different metals in the same solution are tested. The sensitivity and limits of detection of the sensor are determined. As a test for the ability of the chemical reaction to amplify the signal, a grubbs catalyst is added to the solution, and reagents to the lipids, and the sensitivity and limit of detection of the catalyst are tested. Finally, pharmaceuticals and personal care products are added to the water and the sensor array is tested for its ability to detect these contaminants in water.

### REFERENCES

The following references are referred to above and are incorporated herein by reference:
1. S. Lenhert, C. A. Mirkin, H. Fuchs, In situ lipid dip-pen nanolithography under water, Scanning 31, 1-9 (2010).
2. S. Sekula, J. Fuchs, S. Weg-Remers, P. Nagel, S. Schuppler, J. Fragala, N. Theilacker, M. Franzreb, C. Wingren, P. Ellmark, C. A. K. Borrebaeck, C. A. Mirkin, H. Fuchs, S. Lenhert, Multiplexed lipid dip-pen nanolithography on subcellular scales for templating of functional proteins and cell culture, Small 4, 1785-1793 (2008).
3. S. Lenhert, P. Sun, Y. H. Wang, H. Fuchs, C. A. Mirkin, Massively parallel dip-pen nanolithography of heterogeneous supported phospholipid multilayer patterns, Small 3, 71-75 (2007).
4. S. Lenhert, in Introduction to Nanoscale Science and Technology. (NanoInk, Inc., 2010), pp. 163-209.
5. D. T. Chiu, C. F. Wilson, F. Ryttsen, A. Stromberg, C. Farre, A. Karlsson, S. Nordholm, A. Gaggar, B. P. Modi, A. Moscho, R. A. Garza-Lopez, O. Orwar, R. N. Zare, Chemical transformations in individual ultrasmall biomimetic containers, Science 283, 1892-1895 (1999).
6. P. S. Cremer, E. T. Castellana, Solid supported lipid bilayers: From biophysical studies to sensor design, Surface Science Reports 61, 429-444 (2006).
7. A. N. Parikh, J. T. Groves, Materials science of supported lipid membranes, MRS Bulletin 31, 507-512 (2006).
8. M. Tanaka, E. Sackmann, Polymer-supported membranes as models of the cell surface, Nature 437, 656-663 (2005).
9. C. A. Keller, K. Glasmastar, V. P. Zhdanov, B. Kasemo, Formation of supported membranes from vesicles, Physical Review Letters 84, 5443-5446 (2000).
10. K. Jacobson, C. Dietrich, Looking at lipid rafts?, Trends in Cell Biology 9, 87-91 (1999).
11. P. Sharma, R. Varma, R. C. Sarasij, Ira, K. Gousset, G. Krishnamoorthy, M. Rao, S. Mayor, Nanoscale organization of multiple GPI-anchored proteins in living cell membranes, Cell 116, 577-589 (2004).
12. M. Wu, D. Holowka, H. G. Craighead, B. Baird, Visualization of plasma membrane compartmentalization with patterned lipid bilayers, Proc Natl Acad Sci USA 101, 13798-13803 (2004).
13. J. T. Groves, N. Ulman, S. G. Boxer, Micropatterning fluid lipid bilayers on solid supports, Science 275, 651-653 (1997).
14. K. Salaita, P. M. Nair, R. S. Petit, R. M. Neve, D. Das, J. W. Gray, J. T. Groves, Restriction of receptor movement alters cellular response: physical force sensing by EphA2, Science 327, 1380-1385 (2010).
15. K. D. Mossman, G. Campi, J. T. Groves, M. L. Dustin, Altered TCR signaling from geometrically repatterned immunological synapses, Science 310, 1191 (2005).
16. J. Nissen, K. Jacobs, J. O. Radler, Interface dynamics of lipid membrane spreading on solid surfaces, Physical Review Letters 86, 1904-1907 (2001).
17. J. Nissen, S. Gritsch, G. Wiegand, J. O. Radler, Wetting of phospholipid membranes on hydrophilic surfaces: concepts towards self-healing membranes, European Physical Journal B 10, 335-344 (1999).
18. J. Radler, H. Strey, E. Sackmann, Phenomenology and kinetics of lipid bilayer spreading on hydrophilic surfaces, Langmuir 11, 4539-4548 (1995).
19. B. Sanii, A. N. Parikh, Surface-energy dependent spreading of lipid monolayers and bilayers, Soft Matter 3, 974-977 (2007).
20. S. Sekula, J. Fuchs, S. Weg-Remers, P. Nagel , S. Schuppler, J. Fragala, N. Theilacker, M. Franzreb, C. Wingren, P. Ellmark, C. A. K. Borrebaeck, C. A. Mirkin, H. Fuchs, S. Lenhert, Multiplexed lipid dip-pen nanolithography on subcellular scales for templating of functional proteins and cell culture, Small 4, 1785-1793 (2008).
21. S. Lenhert, P. Sun, Y. H. Wang, H. Fuchs, C. A. Mirkin, Massively parallel dip-pen nanolithography of heterogeneous supported phospholipid multilayer patterns, Small 3, 71-75 (2007).
22. B. Sanii, A. N. Parikh, Patterning fluid and elastomeric surfaces using short-wavelength UV radiation and photogenerated reactive oxygen species, Annual Review of Physical Chemistry 59, 411-432 (2008).
23. S. Majd, M. Mayer, Hydrogel stamping of arrays of supported lipid bilayers with various lipid compositions for the screening of drug-membrane and protein-membrane interactions, Angewandte Chemie-International Edition 44, 6697-6700 (2005).
24. J. S. Hovis, S. G. Boxer, Patterning and composition arrays of supported lipid bilayers by microcontact printing, Langmuir 17, 3400-3405 (2001).
25. A. Karlsson, R. Karlsson, M. Karlsson, A. S. Cans, A. Stromberg, F. Ryttsen, O. Orwar, Molecular engineering—Networks of nanotubes and containers, Nature 409, 150-152 (2001).
26. A. Guo, Y. Deng, Y. Wang, B. Holtz, J. Y. Li, N. Traaseth, G. Veglia, B. J. Stottrup, R. Elde, D. Q. Pei, X. Y. Zhu, Fluidic and air-stable supported lipid bilayer and cell-mimicking microarrays, Journal of the American Chemical Society 130, 6267-6271 (2008).
27. Y. F. Dufrene, G. U. Lee, Advances in the characterization of supported lipid films with the atomic force microscope, Biochimica et Biophysica Acta—Biomembranes 1509, 14-41 (2000).
28. Y. Barenholz, D. Gibbes, B. J. Litman, J. Goll, T. E. Thompson, F. D. Carlson, Simple method for preparation of homogeneous phospholipid vesicles, Biochemistry 16, 2806-2810 (1977).
29. F. Szoka, D. Papahadjopoulos, Comparative properties and methods of preparation of lipid vesicles (liposomes), Annual Review of Biophysics and Bioengineering 9, 467-508 (1980).
30. J. Gustafsson, G. Arvidson, G. Karlsson, M. Almgren, Complexes between cationic liposomes and DNA visualized by Cryo-TEM, Biochimica et Biophysica Acta—Biomembranes 1235, 305-312 (1995).
31. T. M. Allen, E. Brandeis, C. B. Hansen, G. Y. Kao, S. Zalipsky, A new strategy for attachment of antibodies to sterically stabilized liposomes resulting in efficient targeting to cancer cells (vol 1237, pg 99, 1995), Biochimica et Biophysica Acta—Biomembranes 1240, 285 (1995).
32. D. Kirpotin, J. W. Park, K. Hong, S. Zalipsky, W. L. Li, P. Carter, C. C. Benz, D. Papahadjopoulos, Sterically stabilized Anti-HER2 immunoliposomes: design and targeting to human breast cancer cells in vitro, Biochemistry 36, 66-75 (1997).
33. D. G. Stamou, S. M. Christensen, Sensing applications of surface-based single vesicle arrays, Sensors 10, 11352-11368 (2010).
34. J. Solon, P. Streicher, R. Richter, F. Brochard-Wyart, P. Bassereau, Vesicles surfing on a lipid bilayer: self-induced haptotactic motion, Proceedings of the National Academy of Sciences of the USA 103, 12382-12387 (2006).
35. M. M. Hanczyc, S. M. Fujikawa, J. W. Szostak, Experimental models of primitive cellular compartments: encapsulation, growth, and division, Science 302, 618-622 (2003).
36. J. W. Szostak, S. S. Mansy, J. P. Schrum, M. Krishnamurthy, S. Tobe, D. A. Treco, Template-directed synthesis of a genetic polymer in a model protocell, Nature 454, 122-125 (2008).
37. T. Oberholzer, M. Albrizio, P. L. Luisi, Polymerase chain-reaction in liposomes, Chemistry & Biology 2, 677-682 (1995).
38. T. Oberholzer, K. H. Nierhaus, P. L. Luisi, Protein expression in liposomes, Biochemical and Biophysical Research Communications 261, 238-241 (1999).
39. J. W. Szostak, D. P. Bartel, P. L. Luisi, Synthesizing life, Nature 409, 387-390 (2001).
40. P. L. Luisi, C. Chiarabelli, P. Stano, Chemical approaches to synthetic biology, Current Opinion in Biotechnology 20, 492-497 (2009).
41. M. B. Eisen, P. T. Spellman, P. O. Brown, D. Botstein, Cluster analysis and display of genome-wide expression patterns, Proceedings of the National Academy of Sciences of the United States of America 95, 14863-14868 (1998).
42. H. Zhu, M. Snyder, Protein chip technology, Current Opinion in Chemical Biology 7, 55-63 (2003).
43. A. Guo, Y. Deng, Y. Wang, B. Holtz, J. Y. Li, N. Traaseth, G. Veglia, B. J. Stottrup, R. Elde, D. Q. Pei, X. Y. Zhu, Fluidic and air-stable supported lipid bilayer and cellmimicking microarrays, Journal of the American Chemical Society 130, 6267-6271 (2008).
44. V. Yamazaki, O. Sirenko, R. J. Schafer, L. Nguyen, T. Gutsmann, L. Brade, J. T. Groves, Cell membrane array fabrication and assay technology, Bmc Biotechnology 5, (2005).
45. A. B. Braunschweig, F. W. Huo, C. A. Mirkin, Molecular printing, Nature Chemistry 1, 353-358 (2009).
46. K. Salaita, Y. H. Wang, C. A. Mirkin, Applications of dip-pen nanolithography, Nature Nanotechnology 2, 145-155 (2007).
47. D. S. Ginger, H. Zhang, C. A. Mirkin, The evolution of dip-pen nanolithography, Angewandte Chemie—International Edition 43, 30-45 (2004).
48. R. D. Piner, J. Zhu, F. Xu, S. H. Hong, C. A. Mirkin, “Dip-pen” nanolithography, Science 283, 661-663 (1999).
49. K. Salaita, Y. H. Wang, J. Fragala, R. A. Vega, C. Liu, C. A. Mirkin, Massively parallel dip-pen nanolithography with 55000-pen two-dimensional arrays, Angewandte Chemie—International Edition 45, 7220-7223 (2006).
50. M. Zhang, D. Bullen, S. W. Chung, S. Hong, K. S. Ryu, Z. F. Fan, C. A. Mirkin, C. Liu, A MEMS nanoplotter with high-density parallel dip-pen manolithography probe arrays, Nanotechnology 13, 212-217 (2002).
51. Y. N. Xia, G. M. Whitesides, Soft lithography, Annual Review of Materials Science 28, 153-184 (1998).
52. C. A. Mirkin, F. W. Huo, Z. J. Zheng, G. F. Zheng, L. R. Giam, H. Zhang, Polymer pen lithography, Science 321, 1658-1660 (2008).
53. S. Sainov, Optical Sensor-Based on Total Internal-Reflection Diffraction Grating, Sensors and Actuators a-Physical 45, 1-6 (1994).
54. J. J. Ramsden, Optical biosensors, Journal of Molecular Recognition 10, 109-120 (1997).
55. X. D. Fan, I. M. White, S. I. Shopoua, H. Y. Zhu, J. D. Suter, Y. Z. Sun, Sensitive optical biosensors for unlabeled targets: A review, Analytica Chimica Acta 620, 8-26 (2008).
56. K. J. Albert, N. S. Lewis, C. L. Schauer, G. A. Sotzing, S. E. Stitzel, T. P. Vaid, D. R. Walt, Cross-reactive chemical sensor arrays, Chemical Reviews 100, 2595-2626 (2000).
57. L. D. Bonifacio, D. P. Puzzo, S. Breslav, B. M. Willey, A. McGeer, G. A. Ozin, Towards the Photonic Nose: A Novel Platform for Molecule and Bacteria Identification, Advanced Materials 22, 1351-+ (2010).
58. L. D. Bonifacio, G. A. Ozin, A. C. Arsenault, Photonic Nose-Sensor Platform for Water and Food Quality Control, Small 7, 3153-3157 (2011).
59. N. P. Chongsiriwatana, J. A. Patch, A. M. Czyzewski, M. T. Dohm, A. Ivankin, D. Gidalevitz, R. N. Zuckermann, A. E. Barron, Peptoids that mimic the structure, function, and mechanism of helical antimicrobial peptides, Proceedings of the National Academy of Sciences of the United States of America 105, 2794-2799 (2008).
60. N. C. Seeman, DNA in a material world, Nature 421, 427-431 (2003).
61. C. A. Mirkin, R. L. Letsinger, R. C. Mucic, J. J. Storhoff, A DNA-based method for rationally assembling nanoparticles into macroscopic materials, Nature 382, 607-609 (1996).
62. W. J. Stark, Nanoparticles in biological systems, Angewandte Chemie—International Edition 50, 1242-1258 (2011).
63. I. L. Medintz, H. T. Uyeda, E. R. Goldman, H. Mattoussi, Quantum dot bioconjugates for imaging, labelling and sensing, Nature Materials 4, 435-446 (2005).
64. M. Sarikaya, C. Tamerler, A. K. Y. Jen, K. Schulten, F. Baneyx, Molecular biomimetics: nanotechnology through biology, Nature Materials 2, 577-585 (2003).
65. L. D. Qin, S. Park, L. Huang, C. A. Mirkin, On-wire lithography, Science 309, 113-115 (2005).
66. A. E. Kusi-Appiah, N. Vafai, P. J. Cranfill, M. W. Davidson, S. Lenhert, Lipid multilayer microarrays for in vitro liposomal drug delivery and screening, Biomaterials 33, 4187-4194 (2012).
67. Y. Wang, L. R. Giam, M. Park, S. Lenhert, H. Fuchs, C. A. Mirkin, A self-correcting inking strategy for cantilever arrays addressed by an inkjet printer and used for dip-pen nanolithography, Small 4, 1666-1670 (2008).
68. O. A. Nafday, S. Lenhert, High-throughput optical quality control of lipid multilayers fabricated by dip-pen nanolithography, Nanotechnology 22, 225301 (2011).
69. T. Mappes, M. Schelb, C. Vannahme, S. Lenhert, B. Rossa, A. Welle, in Proc. of SPIE (2010), vol. 7716 pp. 77162A-77161-77167.
70. O. A. Nafday, T. W. Lowry, S. Lenhert, Multifunctional Lipid Multilayer Stamping, Small 8, 1021-1028 (2012).
71. S. Lenhert, H. Fuchs, C. A. Mirkin, in Nanoprobes, H. Fuchs, Ed. (Wiley-VCH, Weinheim, 2009), vol. 6, pp. 171-196.
72. H. Kirchhoff, S. Lenhert, C. Buchel, L. Chi, J. Nield, Probing the organization of photosystem II in photosynthetic membranes by atomic force microscopy, Biochemistry 47, 431-440 (2008).
73. U. Plutowski, S. S. Jester, S. Lenhert, M. M. Kappes, C. Richert, DNA-based self-sorting of nanoparticles on gold surfaces, Advanced Materials 19, 1951-1956 (2007).
74. X. C. Wu, S. Lenhert, L. F. Chi, H. Fuchs, Interface interaction controlled transport of CdTe nanoparticles in the microcontact printing process, Langmuir 22, 7807-7811 (2006).
75. S. Lenhert, M. Gleiche, H. Fuchs, L. F. Chi, Mechanism of regular pattern formation in reactive dewetting, Chemphyschem 6, 2495-2498 (2005).
76. M. Z. Zhang, S. Lenhert, M. Wang, L. F. Chi, N. Lu, H. Fuchs, N. B. Ming, Regular arrays of copper wires formed by template-assisted electrodeposition, Advanced Materials 16, 409-413 (2004).
77. S. Lenhert, L. Zhang, J. Mueller, H. P. Wiesmann, G. Erker, H. Fuchs, L. F. Chi, Self-organized complex patterning: Langmuir-Blodgett lithography, Advanced Materials 16, 619-624 (2004).
78. H. Kirchhoff, M. Borinski, S. Lenhert, L. F. Chi, C. Buchel, Transversal and lateral exciton energy transfer in grana thylakoids of spinach, Biochemistry 43, 14508-14516 (2004).
79. L. F. Chi, M. Gleiche, S. Lenhert, N. Lu, in Dekker Encyclopedia of Nanoscience and Nanotechnology, J. A. Schwarz, C. Contescu, K. Putyera, Eds. (Marcel Dekker, New York, 2004).
80. N. Lu, M. Gleiche, J. W. Zheng, S. Lenhert, B. Xu, L. F. Chi, H. Fuchs, Fabrication of chemically patterned surfaces based on template-directed self-assembly, Advanced Materials 14, 1812-1815 (2002).
81. C. M. Niemeyer, M. Adler, S. Lenhert, S. Gao, H. Fuchs, L. F. Chi, Nucleic acid supercoiling as a means for ionic switching of DNA-nanoparticle networks, Chembiochem 2, 260-264 (2001).
82. S. Gao, L. Chi, S. Lenhert, B. Anczykowski, C. M. Niemeyer, M. Adler, H. Fuchs, High-quality mapping of DNA-protein complexes by dynamic scanning force microscopy, Chemphyschem 2, 384-388 (2001).
83. C. M. Niemeyer, M. Adler, B. Pignataro, S. Lenhert, S. Gao, L. F. Chi, H. Fuchs, D. Blohm, Self-assembly of DNA-streptavidin nanostructures and their use as reagents in immuno-PCR, Nucleic Acids Research 27, 4553-4561 (1999).
84. D. Jackson, S. Lenhert, A simple method for characterizing iridescence, Florida State Undergraduate Research Journal 1, 39-51 (2011).
85. S. Lenhert, F. Brinkmann, T. Laue, S. Walheim, C. Vannahme, S. Klinkhammer, M. Xu, S. Sekula, T. Mappes, T. Schimmel, H. Fuchs, Nat. Nanotechnol. 5, 275 (2010).
86. E. ten Grotenhuis, W. J. M. vanderKemp, J. G. Blok, J. C. van Miltenburg, J. P. van der Eerden, Colloid Surf B.—Biointerfaces 6, 209 (1996).

All documents, patents, journal articles and other materials cited in the present application are incorporated herein by reference.

While the present invention has been disclosed with references to certain embodiments, numerous modification, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method comprising the following step:
(a) determining that one or more analytes are present in a liquid to which an array of lipid multilayer gratings has been exposed based on scattered light detected by a detector,
wherein each lipid multilayer grating of the array of lipid multilayer gratings comprises iridescent lipid multilayer nanostructures,
wherein the scattered light is formed by scattering one or more incident lights by the array of lipid multilayer gratings,
wherein the scattered light is formed while the lipid multilayer gratings are immersed in the liquid, and
wherein the one or more lipid multilayer gratings comprise ion channels that are activated by the one or more analytes.

2. The method of claim 1, wherein the method comprises the following step:
(b) exposing the array of lipid multilayer gratings to the liquid comprising the one or more analytes.

3. The method of claim 1, wherein step (a) comprises comparing light scattered by the array lipid multilayer gratings and detected by the detector after the array of lipid multilayer gratings is exposed to the liquid to light scattered by the array lipid multilayer gratings and detected by the detector before the array of lipid multilayer gratings is exposed to the liquid.

4. The method of claim 3, wherein the method comprises the following step:
(b) the detector detecting the light scattered by the array lipid multilayer gratings before the array of lipid multilayer gratings is exposed to the liquid.

5. The method of claim 1, wherein a first lipid multilayer grating of the array of lipid multilayer gratings comprises a first lipid, wherein a second lipid multilayer grating of the array of lipid multilayer gratings comprises a second lipid, and wherein the first lipid and the second lipid are different from each other.

6. The method of claim 5, wherein one or more of the lipid multilayer gratings comprises one or more phospholipids.

7. The method of claim 1, wherein the liquid is water.

8. The method of claim 1, wherein step (a) comprises determining a concentration for at least one of the one or more analytes in the liquid.

9. The method of claim 1, wherein step (a) comprises determining that one or more analytes are present in the liquid based on a standard reading for the detector for the liquid.

10. A product comprising:
an array of lipid multilayer gratings on a substrate,
wherein each lipid multilayer grating of the array of lipid multilayer gratings comprises iridescent lipid multilayer microstructures, and
wherein each lipid multilayer structure of the lipid multilayer microstructures comprises one or more ion channels on a surface of the lipid multilayer structure.

11. The product of claim 10, wherein the one or more ion channels are calcium channels.
